SEQUENCE LISTING (118 sequences)

SEQ ID NO: 1 - RNA, 21 nt, Homo sapiens
uuaagacuugcagugauguuu

SEQ ID NO: 2 - RNA, 23 nt, Homo sapiens
ugaggggcagagagcgagacuuu

SEQ ID NO: 3 - RNA, 22 nt, Homo sapiens
uuugguccccuucaaccagcug

SEQ ID NO: 4 - RNA, 22 nt, Homo sapiens
ugucaguuugucaaauacccca

SEQ ID NO: 5 - RNA, 23 nt, Caenorhabditis elegans
uaucacagccagcuuugaugugc

SEQ ID NO: 6 - RNA, 21 nt, Caenorhabditis elegans
ucccugagaccucaaguguga

SEQ ID NO: 7 - RNA, 21 nt, Arabidopsis thaliana
uuuggauugaagggagcucua

SEQ ID NO: 8 - DNA, 71 bp, Homo sapiens
acgggcgagcttttggcccgggttatacctgatgctcacgtataagacgagcaaaaagcttgttggtcaga

SEQ ID NO: 9 - RNA, 22 nt, Homo sapiens
auaagacgagcaaaaagcuugu

SEQ ID NO: 10 - RNA, 77 nt, Homo sapiens
ccucucagggaagcuuuuugcucgaauuauguuucugauccgaauauaagacgaacaaaagguuugucugagggcag

SEQ ID NO: 11 - RNA, 22 nt, Homo sapiens
auaagacgaacaaaagguuugu

SEQ ID NO: 12 - DNA, 16 bp, Homo sapiens
ctttttgctcgtctta

SEQ ID NO: 13 - DNA, 16 bp, Caenorhabditis elegans
tcctagaaagagtaga

SEQ ID NO: 14 - RNA, 22 nt, Homo sapiens
ugugacagauugauaacugaaa

SEQ ID NO: 15 - RNA, 22 nt, Homo sapiens
uggagagaaaggcaguuccuga

SEQ ID NO: 16 - RNA, 23 nt, Homo sapiens
cccaguguucagacuaccuguuc

SEQ ID NO: 17 - RNA, 23 nt, Homo sapiens
caaagugcucauagugcagguag

SEQ ID NO: 18 - RNA, 22 nt, Homo sapiens
aaaccguuaccauuacugaguu

SEQ ID NO: 19 - RNA, 22 nt, Homo sapiens
cagugguuuuacccuaugguag

SEQ ID NO: 20 - RNA, 23 nt, Homo sapiens
caaagugcuguucgugcagguag

SEQ ID NO: 21 - RNA, 21 nt, Homo sapiens
uucacaguggcuaaguuccgc

SEQ ID NO: 22 - RNA, 22 nt, Homo sapiens
uaaugccccuaaaaauccuuau

SEQ ID NO: 23 - RNA, 22 nt, Homo sapiens
ucagugcacuacagaacuuugu

SEQ ID NO: 24 - RNA, 23 nt, Homo sapiens
uaaagugcuuauagugcagguag

SEQ ID NO: 25 - RNA, 21 nt, Homo sapiens
uaaagugcugacagugcagau

SEQ ID NO: 26 - RNA, 22 nt, Homo sapiens
uagcagcacguaaauauuggcg

SEQ ID NO: 27 - RNA, 23 nt, Homo sapiens
uaaggugcaucuagugcagauag

SEQ ID NO: 28 - RNA, 21 nt, Homo sapiens
uucaaguaauucaggauaggu

SEQ ID NO: 29 - RNA, 21 nt, Homo sapiens
cugaccuaugaauugacagcc

SEQ ID NO: 30 - RNA, 22 nt, Homo sapiens
aaaagcuggguugagagggcga

SEQ ID NO: 31 - RNA, 22 nt, Homo sapiens
ugcggggcuagggcuaacagca

SEQ ID NO: 32 - RNA, 23 nt, Homo sapiens
cagugcaauaguauugucaaagc

SEQ ID NO: 33 - RNA, 21 nt, Homo sapiens
caucccuugcaugguggaggg

SEQ ID NO: 34 - RNA, 21 nt, Homo sapiens
uagcagcacagaaauauuggc

SEQ ID NO: 35 - RNA, 22 nt, Homo sapiens
uggagugugacaaugguguuug

SEQ ID NO: 36 - RNA, 22 nt, Homo sapiens
uucaccaccuucuccacccagc

SEQ ID NO: 37 - RNA, 22 nt, Homo sapiens
ucccugagacccuaacuuguga

SEQ ID NO: 38 - RNA, 22 nt, Homo sapiens
uccuguacugagcugccccgag

SEQ ID NO: 39 - RNA, 22 nt, Homo sapiens
ucaggcucaguccccucccgau

SEQ ID NO: 40 - RNA, 22 nt, Homo sapiens
uggacggagaacugauaagggu

SEQ ID NO: 41 - RNA, 23 nt, Homo sapiens
caaagugcuuacagugcagguag

SEQ ID NO: 42 - RNA, 23 nt, Homo sapiens
cgcauccccuagggcauuggugu

SEQ ID NO: 43 - RNA, 24 nt, Homo sapiens
uuuggcaaugguagaacucacacu

SEQ ID NO: 44 - RNA, 22 nt, Homo sapiens
aacauucaaccugucggugagu

SEQ ID NO: 45 - RNA, 22 nt, Homo sapiens
uagcaccauuugaaaucgguua

SEQ ID NO: 46 - RNA, 22 nt, Homo sapiens
cauugcacuugucucggucuga

SEQ ID NO: 47 - RNA, 21 nt, Homo sapiens
uugugcuugaucuaaccaugu

SEQ ID NO: 48 - RNA, 22 nt, Homo sapiens
uguaaacauccucgacuggaag

SEQ ID NO: 49 - RNA, 22 nt, Homo sapiens
uguaaacauccuacacucagcu

SEQ ID NO: 50 - RNA, 22 nt, Homo sapiens
uauugcacuugucccggccugu

SEQ ID NO: 51 - RNA, 22 nt, Homo sapiens
uuauaaagcaaugagacugauu

SEQ ID NO: 52 - RNA, 21 nt, Homo sapiens
uucacaguggcuaaguucugc

SEQ ID NO: 53 - RNA, 22 nt, Homo sapiens
uggcucaguucagcaggaacag

SEQ ID NO: 54 - RNA, 23 nt, Homo sapiens
agcagcauuguacagggcuauga

SEQ ID NO: 55 - RNA, 22 nt, Homo sapiens
gugaaauguuuaggaccacuag

SEQ ID NO: 56 - RNA, 21 nt, Homo sapiens
agcuacaucuggcuacugggu

SEQ ID NO: 57 - RNA, 21 nt, Homo sapiens
uacaguacugugauaacugaa

SEQ ID NO: 58 - RNA, 22 nt, Homo sapiens
ugagguaguagguugugugguu

SEQ ID NO: 59 - RNA, 23 nt, Homo sapiens
ucaagagcaauaacgaaaaaugu

SEQ ID NO: 60 - RNA, 23 nt, Homo sapiens
uguaguguuuccuacuuuaugga

SEQ ID NO: 61 - RNA, 21 nt, Homo sapiens
agggcccccccucaauccugu

SEQ ID NO: 62 - RNA, 22 nt, Homo sapiens
acaggugagguucuugggagcc

SEQ ID NO: 63 - RNA, 22 nt, Homo sapiens
uagguaguuuccuguuguuggg

SEQ ID NO: 64 - RNA, 21 nt, Homo sapiens
ugagaugaagcacuguagcuc

SEQ ID NO: 65 - RNA, 22 nt, Homo sapiens
aaggagcucacagucuauugag

SEQ ID NO: 66 - RNA, 23 nt, Homo sapiens
agcagcauuguacagggcuauca

SEQ ID NO: 67 - RNA, 24 nt, Homo sapiens
ucccugagacccuuuaaccuguga

SEQ ID NO: 68 - RNA, 22 nt, Homo sapiens
uucccuuugucauccuaugccu

SEQ ID NO: 69 - RNA, 23 nt, Homo sapiens
ugugcaaauccaugcaaaacuga

SEQ ID NO: 70 - RNA, 22 nt, Homo sapiens
acaguagucugcacauugguua

SEQ ID NO: 71 - RNA, 22 nt, Homo sapiens
ugagguaguaguuuguacaguu

SEQ ID NO: 72 - RNA, 23 nt, Homo sapiens
aacauucaacgcugucggugagu

SEQ ID NO: 73 - RNA, 22 nt, Homo sapiens
uggcaguguauuguuagcuggu

SEQ ID NO: 74 - RNA, 23 nt, Homo sapiens
uagcaccauuugaaaucaguguu

SEQ ID NO: 75 - RNA, 22 nt, Homo sapiens
aaucguacagggucauccacuu

SEQ ID NO: 76 - RNA, 22 nt, Homo sapiens
cacccguagaaccgaccuugcg

SEQ ID NO: 77 - RNA, 22 nt, Homo sapiens
cagugcaauguuaaaagggcau

SEQ ID NO: 78 - RNA, 22 nt, Homo sapiens
cacgcucaugcacacacccaca

SEQ ID NO: 79 - RNA, 23 nt, Homo sapiens
ucccuguccuccaggagcucacg

SEQ ID NO: 80 - RNA, 23 nt, Homo sapiens
uaagugcuuccauguuuugguga

SEQ ID NO: 81 - RNA, 23 nt, Homo sapiens
guccaguuuucccaggaaucccu

SEQ ID NO: 82 - RNA, 21 nt, Homo sapiens
ucagugcaugacagaacuugg

SEQ ID NO: 83 - RNA, 21 nt, Homo sapiens
cauuauuacuuuugguacgcg

SEQ ID NO: 84 - RNA, 21 nt, Homo sapiens
cauaaaguagaaagcacuacu

SEQ ID NO: 85 - RNA, 22 nt, Homo sapiens
agagguaguagguugcauaguu

SEQ ID NO: 86 - RNA, 23 nt, Homo sapiens
ugugcaaaucuaugcaaaacuga

SEQ ID NO: 87 - RNA, 22 nt, Homo sapiens
uccgucucaguuacuuuauagc

SEQ ID NO: 88 - RNA, 22 nt, Homo sapiens
caaagaauucuccuuuugggcu

SEQ ID NO: 89 - RNA, 21 nt, Homo sapiens
ucacagugaaccggucucuuu

SEQ ID NO: 90 - RNA, 23 nt, Homo sapiens
uuaaugcuaaucgugauaggggu

SEQ ID NO: 91 - RNA, 22 nt, Homo sapiens
ccucccacacccaaggcuugca

SEQ ID NO: 92 - RNA, 22 nt, Homo sapiens
ugagguaggagguuguauaguu

SEQ ID NO: 93 - RNA, 22 nt, Homo sapiens
ucgugucuuguguugcagccgg

SEQ ID NO: 94 - RNA, 22 nt, Homo sapiens
acagcaggcacagacaggcagu

SEQ ID NO: 95 - RNA, 22 nt, Homo sapiens
ugagguaguagguuguaugguu

SEQ ID NO: 96 - RNA, 21 nt, Homo sapiens
gccccugggccuauccuagaa

SEQ ID NO: 97 - RNA, 21 nt, Homo sapiens
aggcaagaugcuggcauagcu

SEQ ID NO: 98 - RNA, 22 nt, Homo sapiens
uucaaguaauccaggauaggcu

SEQ ID NO: 99 - RNA, 23 nt, Homo sapiens
uguaaacauccuacacucucagc

SEQ ID NO: 100 - RNA, 22 nt, Homo sapiens
uuuguucguucggcucgcguga

SEQ ID NO: 101 - RNA, 22 nt, Homo sapiens
uagcaccaucugaaaucgguua

SEQ ID NO: 102 - RNA, 22 nt, Homo sapiens
uguaaacauccccgacuggaag

SEQ ID NO: 103 - RNA, 22 nt, Homo sapiens
ucguaccgugaguaauaaugcg

SEQ ID NO: 104 - RNA, 22 nt, Homo sapiens
cagugcaaugaugaaagggcau

SEQ ID NO: 105 - RNA, 23 nt, Homo sapiens
caacggaaucccaaaagcagcug

SEQ ID NO: 106 - RNA, 22 nt, Homo sapiens
ugagaacugaauuccauggguu

SEQ ID NO: 107 - RNA, 23 nt, Homo sapiens
ucucacacagaaaucgcacccgu

SEQ ID NO: 108 - RNA, 22 nt, Homo sapiens
ucuacagugcacgugucuccag

SEQ ID NO: 109 - RNA, 23 nt, Homo sapiens
agcugguguugugaaucaggccg

SEQ ID NO: 110 - RNA, 20 nt, Homo sapiens
acugccccaggugcugcugg

SEQ ID NO: 111 - RNA, 22 nt, Homo sapiens
uguaaacauccuugacuggaag

SEQ ID NO: 112 - RNA, 23 nt, Homo sapiens
cccaguguuuagacuaucuguuc

SEQ ID NO: 113 - RNA, 22 nt, Homo sapiens
cugugcgugugacagcggcuga

SEQ ID NO: 114 - RNA, 22 nt, Homo sapiens
ucucccaacccuuguaccagug

SEQ ID NO: 115 - RNA, 21 nt, Homo sapiens
acuggacuuggagucagaagg

SEQ ID NO: 116 - RNA, 22 nt, Homo sapiens
aagcugccaguugaagaacugu

SEQ ID NO: 117 - RNA, 22 nt, Homo sapiens
uagcuuaucagacugauguuga

SEQ ID NO: 118 - RNA, 22 nt, Homo sapiens
cuccugacuccagguccugugu

(19) United States
(12) Patent Application Publication
(10) Pub. No.: US 2014/0024700 A1
(43) Pub. Date: Jan. 23, 2014

(54) BLOOD-BORNE MIRNAS AS SURROGATE MARKERS OF DRUG EFFICACY FOR CARDIAC CONDITIONS

(71) Applicants: Eva Van Rooij, Utrecht (NL); Brent Dickinson, Denver, CO (US); Anita Seto, Golden, CO (US)

(72) Inventors: Eva Van Rooij, Utrecht (NL); Brent Dickinson, Denver, CO (US); Anita Seto, Golden, CO (US)

(73) Assignee: miRagen Therapeutics, Inc., Boulder, CO (US)

(21) Appl. No.: 13/994,622

(22) PCT Filed: Dec. 15, 2011
(86) PCT No.: PCT/US2011/065120

§ 371 Date: Oct. 7, 2013

Related U.S. Application Data
(60) Provisional application No. 61/538,585, filed on Sep. 23, 2011.
(60) Provisional application No. 61/495,220, filed on Jun. 9, 2011.
(60) Provisional application No. 61/423,456, filed on Dec. 15, 2010.

(51) Int. Cl. C12Q 1/68; A61K 31/7088; A61K 31/40; G01N 33/53

(52) U.S. Cl. CPC C12Q 1/68; G01N 33/5308; A61K 31/7088; A61K 31/40
USPC 514/44 A; 436/501; 435/6.12; 514/423

(57) ABSTRACT

The present invention provides methods for evaluating or monitoring the efficacy of a therapeutic intervention for treating a cardiac disorder. Such methods comprise measuring or detecting the level of at least one miRNA in a biological sample from a patient receiving the therapeutic intervention and comparing the level to the level of said at least one miRNA in a control sample, wherein the measured level of said at least one miRNA is indicative of the therapeutic efficacy of the therapeutic intervention. Methods of predicting or assessing the severity or progression of heart failure in a patient by measuring one or more miRNAs in a biological sample from the patient are also disclosed.

### PRIORITY

This Application claims priority to U.S. Provisional Application No. 61/538,585, filed Sep. 23, 2011, to U.S. Provisional Application No. 61/495,220, filed Jun. 9, 2011, and to U.S. Provisional Application No. 61/423,456, filed Dec. 15, 2010, each of which are hereby incorporated by reference in their entireties.

### FIELD OF THE INVENTION

The present invention relates to the detection of microRNAs for evaluating or monitoring the efficacy of a therapeutic intervention for cardiac disorders, or for assessing the level of severity or disease progression of heart failure in a patient. The invention further relates to treating patients for cardiac disorders.

### BACKGROUND

Current treatments for cardiac disorders, including heart failure, include pharmacological methods, devices such as the ventricular assist device (VAD), cardiac resynchronization therapy (CRT), and heart transplantation. Pharmacological approaches can include but are not limited to the use of inotropic agents (i.e., compounds that increase cardiac contractility), neurohumoral blockers (e.g., beta-blockers, angiotensin converting enzyme inhibitors), aldosterone antagonists, diuretics, and vasodilators. However, none of these agents is fully effective either alone or in combination. Availability of transplants is highly limited, and since many individuals suffering from heart failure are in poor health, they are frequently not good surgical candidates. For these reasons, heart failure remains a major cause of morbidity and mortality, particularly in the developed world. In addition, it can be difficult to determine the precise etiology of heart failure, a factor impeding the development of more specific therapies. Furthermore, there is a general lack of diagnostic techniques at the molecular level. Thus, there is a need in the art for the discovery of additional diagnostic markers to assist in evaluating the severity of heart failure and its response to treatment, both current and yet to be described. The present invention addresses the foregoing needs, among others.

MicroRNAs (miRNAs, microRNAs or miRs) are a class of regulatory RNAs that post-transcriptionally regulate gene expression. miRNAs are evolutionarily conserved, small non-coding RNA molecules of approximately 18 to 25 nucleotides in length. miRNAs base pair with specific “target” mRNAs and in doing so inhibit translation or promote mRNA degradation (Bartel, Cell, 116, 281-297 (2004)). miRNAs act as repressors of target mRNAs by promoting their degradation, when their sequences are perfectly complementary, or by inhibiting translation, when their sequences contain mismatches.

MicroRNAs have been implicated in a number of biological processes including regulation and maintenance of cardiac function (see, Eva Van Rooij and Eric Olson, MicroRNAs: Powerful new regulators of heart disease and proactive therapeutic targets, J. Clin. Invest. 117(9):2369-2376 (2007); and Chien K R, Molecular Medicine: MicroRNAs and the tell-tale heart, Nature 447, 389-390 (2007)). miRNAs have also been reported to be involved in the development of organisms (Ambros, Cell 113: 673-676) and are differentially expressed in numerous tissues (Xu et al., 2003 Curr. Biol. 13:790-795; Landgraf et al., 2007 Cell 129:1401-14), in viral infection processes (Pfeffer et al., 2004 Science 304: 734-736), and associated with oncogenesis (Calin, et al., 2004 Proc. Natl. Acad. Sci. USA 101: 2999-3004; Calin et al., 2002, Proc. Natl. Acad. Sci. USA 99(24): 15524-15529). Therefore, miRs represent a relatively new class of therapeutic targets for conditions such as cardiac hypertrophy, myocardial infarction, heart failure, vascular damage, and pathologic cardiac fibrosis, among others. The present invention is based in part on the surprising discovery that detection of miRNAs is useful for evaluating and monitoring the therapeutic efficacy for treatment of a cardiac disorder, and for guiding treatment decisions.

### SUMMARY OF THE INVENTION

The present invention provides methods for evaluating or monitoring the efficacy of a therapeutic intervention for treating a cardiac disorder. Such methods comprise measuring or detecting the level of at least one miRNA (or detecting a panel of miRNAs) in a biological sample from a patient receiving the therapeutic intervention. In some embodiments, the level of the at least one miRNA in the biological sample is compared to a reference level, or the measured level of the at least one miRNA in a control sample. The measured level of said at least one miRNA is indicative of the therapeutic efficacy of the therapeutic intervention. In some cases, an increase or decrease in the level of the miRNA is indicative of the efficacy of the therapeutic intervention. In some embodiments, the at least one miRNA is selected from a miRNA listed in any of Tables 2-4. In some embodiments, a plurality of miRNAs from Tables 2-4 are measured, and compared to reference levels or levels in control samples, so as to assess or monitor the therapy. Where a panel of miRNAs are determined in the patient sample, the patient sample may be classified as indicative of effective or non-effective intervention on the basis of a classifier algorithm. In some embodiments, the therapeutic intervention is a chemically-modified antisense oligonucleotide targeting miR-208a and/or miR-208b or a standard of care therapy, such as angiotensin-converting enzyme (ACE) inhibitor. Based on the measured miRNAs levels, therapy may be continued or altered, e.g., by change of dose or dosing frequency, or by addition of other active agents, or change of therapeutic regimen altogether.

In some embodiments, a change in the measured level of the at least one miRNA relative to a sample from the patient taken prior to treatment or earlier during the treatment regimen is indicative of the therapeutic efficacy of the therapeutic intervention.

In some embodiments, the cardiac disorder is a myocardial infarction, pathologic cardiac hypertrophy, heart failure, or hypertension.

The present invention also provides methods for altering the treatment regimen of a therapeutic entity comprising detecting the level of at least one miRNA in a biological sample from a patient receiving the therapeutic intervention and altering the treatment regimen based on an increase or decrease in the level of the at least one miRNA in said biological sample.

The present invention further provides methods of providing useful information for evaluating or monitoring the efficacy of a therapeutic intervention for treating a cardiac disorder comprising determining the level of at least one miRNA in a biological sample of a patient and providing the level of the at least one miRNA to an entity that provides a determination of the efficacy based on an increase or decrease in the level of the at least one miRNA.

The present invention also encompasses a method of predicting or assessing the level of severity of heart failure or heart failure progression in a patient. In one embodiment, the method comprises measuring the level of at least one miRNA selected from Table 1 in a biological sample from a patient; and comparing the measured level to a reference level or the level of said at least one miRNA in a control sample, wherein the measured level of said at least one miRNA is indicative of the level of severity of heart failure or heart failure progression in the patient. In some embodiments, an increase or decrease in the level of the miRNA is indicative of the level of severity of heart failure or heart failure progression in the patient.

In still other aspects, the invention provides a method for treating a patient for heart failure, the patient having elevated levels (e.g., serum or plasma levels) of one or more of miR-223, miR-16, miR-93, miR-106(b), and/or miR-423-5p, or other combination of up-regulated or down-regulated markers (e.g., combination of at least two) listed in Table 1. In accordance with this aspect, the patient is treated for heart failure with conventional therapy (such as an ACE inhibitor), or with an anti-miR therapeutic strategy described herein (e.g., anti-miR-208(b)). The levels of miR-223, miR-16, miR-93, miR-106(b) and/or miR-423-5p may be determined prior to treatment, and/or during treatment to monitor efficacy of the regimen. In some embodiments, the treatment is anti-miR-208(b), and the level of miR-19(b) or a combination of markers listed in one or more of Tables 2-4 is monitored during treatment.

Another aspect of the invention is a kit containing a reagent for measuring at least one miRNA in a biological sample, instructions for measuring the at least one miRNA and instructions for evaluating or monitoring the efficacy of a therapeutic intervention for treating a cardiac disorder in a patient based on the level of the at least one miRNA. In some embodiments, the kit contains reagents for measuring from two to about twenty human miRNAs, including at least one, two, three, or more from Tables 2-4. Kits for assessing or predicting the severity or progression of heart failure in a subject may comprise a reagent for measuring at least one miRNA in a biological sample and instructions for assessing heart failure severity or progression based on the level of the at least one miRNA are also included in the invention. In some embodiments, the kit reagent comprises a miRNA-specific primer and/or probe for reverse transcribing, amplifying, and/or hybridizing to one or more miRNAs described herein. Such kits can further comprise one or more normalization controls and/or a TaqMan probe specific for each miRNA of the kit.

### DESCRIPTION OF THE FIGURES

FIG. 1. Therapeutic silencing of miR-208a is beneficial during heart failure. A. Kaplan-Meier survival curves in the Dahl hypertensive rat model show a pronounced decrease in survival in response to an 8% high-salt (HS) diet for both HS/saline and HS/control groups, which is significantly improved in response to antimiR-208a treatment. Rats were dosed every 2 weeks at 25 mg/kg starting 1 week after the HS diet. B. Body weight analysis indicates that Dahl hypertensive rats on an 8% HS diet exhibit reduced weight gain compared to animals on a low-salt (LS) diet, whereas HS/antimiR-208a-treated rats show a significantly better maintenance in weight gain. For A and B, n=6 for LS/Saline; n:=15 for HS/Saline and HS/Control; and n=14 for HS/antimiR-208a. The “n” on the graph represents total survivors remaining at week 8 after the diet. C. Body weight analysis of Dahl rats on the 4% HS diet shows significant reductions in weight gain compared to LS diet controls, whereas both 5 and 25 mg/kg injections every 2 weeks are sufficient to maintain weight gain comparable to that in animals on a normal diet. D. Echocardiography measurements indicate that the increase in isovolumic relaxation time (IVRT) and decrease in mitral valve early to active filling velocity ratio (MV E/A) in response to 4% HS diet are significantly improved in response to antimiR-208a treatment 8 weeks after the start of the diet. For C and D, n=10 for all groups. E. Representative images of hematoxylin and eosin (H&E)- and Picrosirius Red-stained left ventricular histological sections indicate an increase in cardiomyocyte hypertrophy and perivascular fibrosis in response to the 4% HS diet for 8 weeks, whereas both parameters are reduced in response to antimiR-208a treatment. F. Bar-graph representation of histological quantification showing significantly less hypertrophy and fibrosis in the presence of antimiR-208a. In D and F, error bars depict SEM, * p<0.05 vs. HS saline, #p<0.05 vs. LS saline.

FIG. 2. AntimiR-208a treatment reduces miR-499 and Myh7 in Dahl salt-sensitive rats. All analyses were performed 8 weeks after the start of a 4% high-salt (HS) diet and 7 weeks after the start of antimiR treatment (n=10 for all groups in A and C). In B and D, each lane is a representative animal from the n===10 group. Rats were dosed every 2 weeks at 25 mg/kg (control) or the indicated dose of anti-miR-208a starting 1 week after the HS diet. Red lines on graphs separate the groups. A. Real-time PCR analysis indicates a dose-dependent reduction of miR-208a in both left ventricle (LV) and right ventricle (RV), which corresponds to a dose-dependent reduction in miR-499. Although miR-208b is increased in response to the HS diet, antimiR-208a significantly blunts this response. Administration of a scrambled control chemistry had no effect on the expression of miR-208a, miR-499 or miR-208b, B. Regulation of miR-499 and miR-208b in response to antimiR-208a treatment shown by Northern blot analysis. C. Real-time PCR analysis shows that HS diet reduces Myh6 and increases Myh7. AntimiR-208a treatment dose-dependently increases Myh6 expression and reduces Myh7b expression. The HS diet-induced increase in Myh7 is dose-dependently reduced by antimiR-208a. D. Western blot analysis for Myh7 from ventricular tissue confirms the dose-dependent reduction in response to antimiR-208a treatment. GAPDH is used as a loading control. E. Quantification of HP1β Western blot showing miR-208a target depression in the presence of anti-miR-208a. In A and C, error bars depict SEM, * p<0.05 vs. HS saline, #p<0.05 vs. LS saline.

FIG. 3. miR-499 in plasma serves as a biomarker for antimiR-208a efficacy. A. Real-time PCR analysis on plasma samples indicates an increase in miR-499 in response to high-salt (HS) diet, whereas antimiR-208a significantly lowers the detection of miR-499 in plasma 8 weeks after the start of a 4% HS diet and 7 weeks after the onset of antimiR treatment. B. Further miRNA analysis additionally indicates a decrease in plasma detectable miR-423-5p in response to antimiR-208a. RNA was isolated from plasma samples using Trizol-LS with glycogen assisted RNA precipitation (Invitrogen) and RT-PCR was performed using commercial miRNA Taqman assays from Applied Biosystems. Prior to RNA purification, 5 fmol of synthetic C. Elegans lin-4 and miR-2 were added to samples and plasma miRNA values were normalized to the average recovery of synthetic input sequences. *P<0.05 vs HS saline (n=10 per group).

FIG. 4. Food intake of Dahl salt-sensitive rats. Based on ingested amount of diet, there are no significant differences between the treatment groups, excluding that the differences in weight gain and health are due to differences in intake of the HS diet in the Dahl hypertensive rats. Week 4 (A), Week 5 (B), Week 6 (C) and Week 7 (D) are shown.

FIG. 5. Plasma detection of muscle specific miRNAs. Plasma detection of miR-208a (A) and miR-1 (B), two muscle-specific miRNAs, reveals that levels of these miRNAs show no response to antimiR-208a treatment in Dahl hypertensive rats.

FIG. 6. Cardiac tissue markers demonstrating molecular efficacy following antimiR treatment. All analyses were performed 8 weeks after the start of a 4% high-salt (HS) diet and 7 weeks after the start of antimiR treatment. A. Real-time PCR analysis for miR-208a in cardiac tissue in the indicated groups. B. Real-time PCR analysis shows that HS diet reduces Myh6 expression and antimiR-208a treatment prevents this decrease. Treatment with the control oligonucleotide has no effect on the HS diet-induced decrease in Myh6 expression. C. Real-time PCR analysis shows that HS diet increases Myh7 expression, and treatment with an antimiR-208a oligonucleotide reduces this increase.

FIG. 7. Time course data showing plasma miRNA levels as measured by real time PCR for miR-16 (A), miR-19b (B), miR-423-5p (C), and miR-499 (D) with disease progression in rats with hypertension-induced heart failure treated with antimiR-208a oligonucleotide (10101), control oligonucleotide (10591), or saline.

FIG. 8. Line graphs depicting body weight versus weeks on high-salt diet for each of the indicated treatment groups. Panel A shows body weight over time for all treatment groups whereas panel B shows a subset of the treatment groups. # indicates as significance of p≦0.05 by ANOVA. Body weight graphs were generated by carrying over final body weights of those rats that died prior to the end of the study.

FIG. 9. AntimiR-208a treatment improves survival in Dahl salt-sensitive rats on a high-salt diet. Kaplan-Meier survival curves in the Dahl hypertensive rat model exposed to the indicated treatments. A pronounced decrease in survival is observed in response to a 6% high-salt (HS) diet for both HS/saline and HS/1.5 mg/kg Captopril groups, which is significantly improved in response to antimiR-208a (M−10101) treatment. No mortality was observed in the low-salt (LS) controls or HS/M-10101-treated rats. * p<0.05 vs M-10101 alone.

FIG. 10. Real time PCR analysis of miR-208a (A), miR-499 (B) and miR-208b (C) in cardiac tissue in rats in the indicated treatment groups. #p<0.05 vs. LS/saline, *p<0.05 vs. HS/saline.

FIG. 11. Real time PCR analysis of αMHC (Myh6, A), Myh7b (B), and βMHC (Myh7, C) in cardiac tissue in rats in the indicated treatment groups. #p<0.05 vs. LS/saline, *p<0.05 vs. HS/saline.

FIG. 12. Real time PCR analysis of direct target genes of miR-208, Dynlt1 (A), Vcpip (B), and Tegt (C), in cardiac tissue in rats in the indicated treatment groups. #p<0.05 vs. LS/saline, *p<:0.05 vs. HS/saline.

FIG. 13. Real time PCR analysis of plasma levels of miR-423-5p (A), miR-106b (B), miR-16 (C), miR-92a (D), miR-378 (E) miR-210 (F), miR-378* (G), miR-20b (H), and miR-93 (I) in rats in the indicated treatment groups. Plasma levels of these miRNAs correlated with efficacy of both antimiR-208a and captopril treatments.

FIG. 14. Real time PCR analysis of plasma levels of miR-19b (A), miR-223 (B), miR-21 (C), and miR-150 (D) in rats in the indicated treatment groups. Plasma levels of miR-19b and miR-223 appeared to correlate with efficacy of antimiR-208a olignucleotide therapy whereas plasma levels of miR-21 and miR-150 appeared to correlate with efficacy of captopril therapy.

### DETAILED DESCRIPTION OF THE INVENTION

The methods of the present invention are based, in part, on the surprising discovery that the level of one or more miRNAs can be used as an indicator of the therapeutic efficacy of a therapeutic intervention for cardiac disorders or assessing the severity or disease progression of cardiac disorders, such as heart failure. In some embodiments, the present invention provides methods for evaluating and/or monitoring the efficacy of a therapeutic intervention for treating a cardiac disorder. These methods can include the step of measuring the level of at least one miRNA in a biological sample from a patient receiving a therapeutic intervention and comparing the measured level to a reference level or the level of at least one miRNA in a control sample. The measured level of the at least one miRNA is indicative of the therapeutic efficacy of the therapeutic intervention. In certain embodiments, the method further comprises altering the therapeutic intervention based on the measured level of said at least one miRNA in the biological sample.

Accordingly, one aspect of the methods of the present invention is providing for measuring or detecting the level of at least one miRNA in a biological sample. In some embodiments, the microRNA whose level is measured or detected is an intron-embedded miRNA. In some embodiments, the microRNA whose level is measured or detected is expressed in heart tissue. In particular embodiments, the microRNA whose level is measured is selected from a microRNA listed in any one of Tables 1-4. In certain embodiments, the level of each microRNA in a panel of microRNAs selected from any one of Tables 2-4 is measured. For instance, in some embodiments of the method, two or more, three or more, four or more, five or more, ten or more, fifteen or more, twenty or more, twenty five or more, thirty or more, thirty five or more, forty or more, fifty or more, sixty or more, seventy or more, eighty or more, or ninety or more microRNAs selected from one or more of Tables 1-4 are measured. In particular embodiments, the level of one or more microRNAs or a panel of microRNAs selected from Table 4 are measured.

In some embodiments, a panel of less than twenty, less than fifteen, less than ten, or less than five miRNAs are tested, the panel including one, two, three, four, or five miRNAs from any one of Tables 1-4. Where the patient is suspected of having heart failure, and/or suspected of being in need of therapy, the panel may comprise miRNAs listed in Table 1. Where the patient is undergoing therapy for heart failure, the panel may comprise miRNAs selected from Table 2, 3, or 4.

Measuring or detecting the amount of microRNA in a sample can be performed in any manner known to one skilled in the art and such techniques for measuring or detecting the level of an miRNA are well known and can be readily employed. A variety of methods for detecting miRNAs have been described and include Northern blotting, microarrays, electrochemical methods (oxidation of miRNA-ligated nanoparticles), bioluminescent, bio luminescent protein reassembly, BRET-based (B RET: bioluminescence resonance energy transfer), RT-PCR, fluorescence correlation spectroscopy and surface-enhanced Raman spectroscopy (see, e.g., Cissell, K. A. and Deo, S. K., Trends in microRNA detection, Anal. Bioanal. Chem., 394:1109-1116 (2009); incorporated herein by reference in its entirety).

There are also commercially available kits, such as the qRT-PCR miRNA Detection Kit available from Ambion, U.S.A., which can be used for detecting and quantifying microRNA using quantitative reverse transcriptase polymerase chain reaction. TaqMan MicroRNA Assays, which employ a target-specific stern-loop reverse transcription primer to compensate for the short length of the mature miRNA, is also available from Applied Biosystems (Life Technologies, Inc., USA). qSTAR MicroRNA Detection Assays, commercially available from OriGene, Inc. (USA), can also be used.

Other commercially available kits, such as PAXgene Blood miRNA Kit (which uses silica-based RNA purification technology) can be employed for isolating miRNAs of 18 nucleotides or longer, available from Qiagen, USA. The miScript PCR System, a three-component system which converts miRNA and mRNA into cDNA and allows for detection of miRNAs using SYBR Green-based real-time PCR, can be employed for quantification of mature miRNA, precursor miRNA, and mRNA all from a single sample (also available from Qiagen, USA). GeneCopoeia has a commercial kit available that is based on using RT-PCR in conjunction with SYBR Green for quantitation of miRNA (All-in-One™ miRNA qRT-PCR Detection Kit, available from GeneCopoeia, Inc., USA). mirVANA, available from Life Technologies, Inc. (USA), employs glass fiber filter (GFF)-based method for isolating small RNAs.

The methods for detecting the miRNA can also include hybridization-based technology platforms and massively-parallel next generation small RNA sequencing that allow for detection of multiple microRNAs simultaneously. One commercially-available hybridization-based technology utilizes a sandwich hybridization assay with signal amplification provided by a labeled branched DNA (Panornics). Another hybridization-based technology is available from Nanostring Technology (nCounter miRNA Expression Assay), where multiple miRNA sequences are detected and distinguished with fluorescently-labeled sequence tags. Examples of next-generation sequencing are available from Life Technologies (SOLiD platform) and Illumina, Inc.

miRNAs can also be isolated by methods described in the art for isolating small RNA molecules (see, e.g., U.S. Patent Publication No. 20100291580, U.S. Patent Publication No. 20100222564, U.S. Patent Publication No. 20060019258, U.S. Patent Publication No. 20110054009 and U.S. Patent Publication No. 20090023149; all of which are incorporated herein by reference in their entireties).

Other methods for isolation of miRNA from a sample include employing a method comprising the following steps: a) obtaining a sample having an miRNA; b) adding an extraction solution to the sample; c) adding an alcohol solution to the extracted sample; d) applying the sample to a mineral or polymer support; and, e) eluting the RNA containing the miRNA from the mineral or polymer support with an ionic solution. Other procedures for isolating miRNA molecules from a sample can involve: a) adding an alcohol solution to the sample; b) applying the sample to a mineral or polymer solid support; c) eluting miRNA molecules from the support with an ionic solution; and, d) using or characterizing the miRNA molecules. (See, U.S. Patent Publication No. 20100222564; incorporated herein by reference in its entirety.)

miRNA can also be isolated by methods involving separation of miRNA from mRNA, such as those described in U.S. Patent Publication No. 20060019258; incorporated herein by reference in its entirety. These methods comprise the steps of a) providing a biological isolate including mRNA having a 5′ cap structure and small RNA having a 5′ phosphate; b) contacting the isolate with a phosphate reactive reagent having a label moiety under conditions wherein the label moiety is preferentially added to the 5′ phosphate over the 5′ cap structure, thereby producing labeled small RNA; and c) distinguishing the small RNA from the mRNA according to the presence of the label.

Examples of methods of isolating and/or quantifying microRNAs can also include but are not limited to hybridizing at least a portion of the microRNA with a fluorescent nucleic acid (a fluorescent probe), and reacting the hybridized microRNA with a fluorescent reagent, wherein the hybridized microRNA emits a fluorescent light or hybridizing at least a portion the microRNA to a radio-labeled complementary nucleic acid. There are commercially available products for fluorescent labeling and detection of miRNAs. NCode miRNA Rapid Labeling System and NCode Rapid Alexa Fluor 3 miRNA Labeling System are both commercially available from Life Technologies, Inc. (USA). Furthermore, fluorescent labels are commercially available and can include the Molecular Probes Alexa Flour dyes, available from Life Technologies, Inc. (USA), including Alexa Fluor 3, Alexa Fluor 5, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and Alexa Fluor 750. Lumiprobes Cy dyes, including Cy2, Cy3, Cy3B, Cy3.5, Cy5, Cy5.5 and Cy7. The DyLight fluorophores, available from ThermoScientific (USA), including DyLight 350, DyLight 405, DyLight 488, DyLight 550, DyLight 594, DyLight 633, DyLight 650, DyLight 680, DyLight 750 and DyLight 800. FluoProbes include FluoProbes 390, FluoProbes 488, FluoProbes 532, FluoProbes 547H, FluoProbes 594, FluoProbes 647H, FluoProbes 682, FluoProbes 752 and FluoProbes 782.

Locked nucleic acid probes can also be employed. For example, the miRCURY LNA microRNA ISH Optimization Kits (FFPE) provides for detection of microRNAs. This kit employs double DIG*-labeled miRCURY LNA™ microRNA Detection that can be used for in situ hybridization and is commercially available from Exiqon (USA and Denmark).

In some embodiments a probe for detecting an miRNA can include a single-stranded molecule, including a single-stranded deoxyribonucleic acid molecule, a single-stranded ribonucleic acid molecule, a single-stranded peptide nucleic acid (PNA), or a single-stranded locked nucleic acid (LNA). In some embodiments the probe is substantially complementary, for example 80%, 85%, 90%, 95%, 96%, 97%, 98%, 99% or 100% identical to the complement of the miRNA being detected, such that the probe is capable of detecting the miRNA. In some embodiments, the probe is substantially identical, 80%, 85%, 90%, 95%, 96%, 97%, 98%, 99%, or 100% identical to the miRNA, such that the probe is capable of detecting the complement of the miRNA. In some instances the probe is at least 5 nucleotides, at least 10 nucleotides, at least 15 nucleotides, at least 20 nucleotides, at least 25 nucleotides, at least 30 nucleotides or at least 40 nucleotides. In some cases, the probe may be no longer than 25 nucleotides, no longer than 35 nucleotides; no longer than 50 nucleotides; no longer than 75 nucleotides, no longer than 100 nucleotides or no longer than 125 nucleotides in length. In some embodiments the probe is substantially complementary to or substantially identical to at least 5 consecutive nucleotides of the miRNA, for example at least 6, 7, 8, 9, 10, 11, 12, 13, 14, 15, 16, 17, 18, 20, 21 and 22, or more consecutive nucleotides. In some embodiments, the probe can be 5-20, 5-25, 5-50, 50-100, or over 100 consecutive nucleotides long.

In some preferred embodiments, the methods for detecting the miRNA include real-time PCR, miRNA Taqman array cards, and Northern blot analysis. In some embodiments, the miRNAs detected include hsa-miR-542-3p, hsa-miR-185, hsa-miR-199a-5p, hsa-miR-20b, hsa-miR-423-5p, hsa-miR-451, hsa-miR-140-5p, hsa-miR-93, hsa-miR-27a, hsa-miR-365, hsa-miR-148a, hsa-miR-20a, hsa-miR-133a, hsa-miR-106b, hsa-miR-16, hsa-miR-18a, hsa-miR-26b, hsa-miR-192, hsa-miR-320a, hsa-miR-223, hsa-miR-744, hsa-miR-301a, hsa-miR-188-5p, hsa-miR-195, hsa-miR-122, hsa-miR-197, hsa-miR-125b-5p, hsa-miR-486-5p, hsa-miR-484, hsa-miR-184, hsa-miR-17, hsa-miR-324-5p, hsa-miR-182, hsa-miR-181c, hsa-miR-29c, hsa-miR-25, hsa-miR-218, hsa-miR-30a, hsa-miR-30b, hsa-miR-92a, hsa-miR-340-5p, hsa-miR-27b, hsa-miR-24, hsa-miR-103a, hsa-miR-203, hsa-miR-222, hsa-miR-101a, hsa-let-7b, hsa-miR-335-5p, hsa-miR-142-3p, hsa-miR-296-5p, hsa-miR-125a-3p, hsa-miR-196b, hsa-miR-143, hsa-miR-28, hsa-miR-107, hsa-miR-125a-5p, hsa-miR-204, hsa-miR-19b, hsa-miR-199a-3p, hsa-let-7g, hsa-miR-181a, hsa-miR-449a, hsa-miR-29b, hsa-miR-487b, hsa-miR-99b, hsa-miR-130a, hsa-miR-574-3p, hsa-miR-339-5p, hsa-miR-302a, hsa-miR-145, hsa-miR-152, hsa-miR-126-5p (miR-126*), hsa-miR-142-5p, hsa-let-7d, hsa-miR-19a, hsa-miR-340-3p, hsa-miR-186, hsa-miR-128a, hsa-miR-155, hsa-miR-532-3p, hsa-let-7e, hsa-miR-187, hsa-miR-214, hsa-let-7c, hsa-miR-331-3p, hsa-miR-31, hsa-miR-26a, hsa-miR-30c, hsa-miR-375, hsa-miR-29a, hsa-miR-30d, hsa-miR-126, hsa-miR-130b, hsa-miR-191, hsa-miR-146a, hsa-miR-342-3p, hsa-miR-139-5p, hsa-miR-138, hsa-miR-324-3p, hsa-miR-30e, or combinations thereof. In other embodiments, the miRNAs detected include hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-126, hsa-miR-133a, hsa-miR-499, hsa-miR-19b, hsa-miR-199b-5p, hsa-miR-204, hsa-miR-145, hsa-miR-195, hsa-miR-125a-5p, hsa-miR-143, hsa-miR-214, or any combination thereof. In still other embodiments, the miRNAs detected include miR-423-5p, miR-106b, miR-16, miR-92a, miR-378, miR-210, miR-378*, miR-20b, miR-93, miR-19b, miR-223, miR-21, miR-150, or combinations thereof.

In order to be used as an indicator of the therapeutic efficacy of a therapeutic intervention for cardiac disorders, the level of the miRNA must be determined. The amount of microRNA in a patient sample can be compared to a standard amount of the microRNA present in patients with a cardiac disorder (e.g., heart failure) or in the healthy population (e.g., each of which may be referred to as a reference level). In other embodiments, the level is compared to the amount of microRNA in a control sample (a sample not from a cardiac disorder subject) or compared to the amount of the microRNA in a sample taken from a patient prior to treatment with a therapeutic intervention or a sample taken from an untreated patient. Standard levels for a microRNA can be determined by determining the level of a microRNA in a sufficiently large number of samples obtained from normal, healthy control subjects to obtain a pre-determined reference or threshold value. As used herein, “reference value” refers to a pre-determined value of the level or concentration of a miRNA ascertained from a known sample.

A standard level can also be determined by determining the level of the microRNA in a sample from a patient prior to treatment with the therapeutic intervention. Further, standard level information and methods for determining standard levels can be obtained from publically available databases, as well as other sources. (See, e.g., Bunk, D. M., “Reference Materials and Reference Measurement Procedures: An Overview from a National Metrology Institute,” Clin. Biochem. Rev., 28(4):131-137 (2007); and Remington: The Science and Practice of Pharmacy, Twenty First Edition (2005).) In some embodiments, a known quantity of another miRNA that is not normally present in the sample is added to the sample (i.e. the sample is spiked with a known quantity of exogenous miRNA) and the level of one or miRNAs of interest is calculated based on the known quantity of the spiked miRNA. The comparison of the measured levels of the one or more miRNAs to a reference amount or the level of one or more of the miRNAs in a control sample can be done by any method known to a skilled artisan. For example, comparing the amount of the microRNA in a sample to a standard amount can include comparing the ratio between 5S rRNA (or the spiked miRNA) and the miRNA in a sample to a published or known ratio between 5S rRNA (or the spiked miRNA) and the miRNA in a control sample. In instances when the amount of microRNA is compared to a control, the control sample may be obtained from any source known to not be affected by a cardiac disorder. The level of miRNA in a sample taken from a patient after administration of a therapeutic intervention can also be compared to the level of miRNA in a sample taken from the patient prior to administration of a therapeutic intervention.

According to the present invention, in some embodiments a difference (increase or decrease) in the measured level of the miRNA relative to the level of the miRNA in the control sample (e.g., sample in patient prior to treatment or an untreated patient) or a pre-determined reference value is indicative of the therapeutic efficacy of the therapeutic intervention. In some embodiments, an increase in the measured level of the miRNA relative to the level of the miRNA in the control sample or pre-determined reference value is indicative of the therapeutic efficacy of the therapeutic intervention. For instance, in such embodiments, when the level of one or more miRNAs selected from an miRNA listed in Table 3 or 4 is increased when compared to the level in a control sample or pre-determined reference value in response to a therapeutic intervention, the increase is indicative of therapeutic efficacy of the therapeutic intervention. In certain embodiments, when the level of one or more miRNAs selected from hsa-miR-19b, hsa-miR-199b-5p, hsa-miR-204, hsa-miR-145, hsa-miR-195, hsa-miR-125a-5p, hsa-miR-143, and hsa-miR-214 is increased when compared to the level in a control sample or pre-determined reference value in response to a therapeutic intervention, the increase is indicative of therapeutic efficacy of the therapeutic intervention.

According to the present invention, in some embodiments a reduction or decrease in the measured level of the miRNA relative to the level of the miRNA in the control sample (e.g., sample in patient prior to treatment or an untreated patient) or pre-determined reference value is indicative of the therapeutic efficacy of the therapeutic intervention. For instance, in such embodiments, when the level of one or more miRNAs selected from an miRNA listed in Tables 2 or 4 is decreased when compared to the level in a control sample or pre-determined reference value in response to a therapeutic intervention, the decrease is indicative of therapeutic efficacy of the therapeutic intervention. In certain embodiments, when the level of one or more miRNAs selected from hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-126, hsa-miR-133a, and hsa-miR-499 is decreased when compared to the level in a control sample or pre-determined reference value in response to a therapeutic intervention, the decrease is indicative of therapeutic efficacy of the therapeutic intervention.

Sampling methods are well known by those skilled in the art and any applicable techniques for obtaining biological samples of any type are contemplated and can be employed with the methods of the present invention. (See, e.g., Clinical Proteomics: Methods and Protocols, Vol. 428 in Methods in Molecular Biology, Ed. Antonia Vlahou (2008).) Samples can include any biological sample from which miRNA can be isolated. Such samples can include serum, blood, plasma, whole blood and derivatives thereof, cardiac tissue, skin, hair, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, cardiac tissue, as well as other tissue extract samples or biopsies. In some embodiments, the biological sample is plasma or serum. In other embodiments, the biological sample is cardiac tissue.

The biological sample for use in the disclosed methods can be obtained from the patient at any point following the start of the therapeutic intervention. In some embodiments, the sample is obtained at least 1, 2, 3, or 6 months following the start of the therapeutic intervention. In some embodiments, the sample is obtained least 1, 2, 3, 4, 6 or 8 weeks following the start of the therapeutic intervention. In some embodiments, the sample is obtained at least 1, 2, 3, 4, 5, 6, or 7 days following the start of the therapeutic intervention. In some embodiments, the sample is obtained at least 1 hour, 6 hours, 12 hours, 18 hours or 24 hours after the start of the therapeutic intervention. In other embodiments, the sample is obtained at least one week following the start of the therapeutic intervention. In some embodiments, one or more miRNAs selected from any one of Tables 2-4 is measured between 1 and 8 weeks following administration of a miRNA-based therapeutic, such as an antisense oligonucleotide targeting a miRNA (e.g. anti-miR treatment). In some embodiments, one or more of the miRNAs is measured in plasma samples between 2 and 7 weeks after an anti-miR treatment. In some embodiments, one or more of the miRNAs is measured in plasma samples at 1, 2, 3, 4, 5, 6, 7 or 8 weeks after an anti-miR treatment. In some embodiments, one or more of the miRNAs is measured in plasma samples at 7 weeks after an anti-miR treatment. In some embodiments, one or more of the miRNAs is measured in plasma samples at 2 weeks after an anti-miR treatment.

For detecting microRNAs as contemplated by the methods of the present invention, the samples for analysis may contain, in some embodiments, between about 1 ng and about 100 ng of RNA. In some embodiments, the sample contains between about 10 ng and about 90 ng of RNA. In some embodiments, the sample contains between about 20 ng and about 80 ng. For detecting microRNAs as contemplated by the methods of the present invention, the samples for analysis may also contain between 0.1 and 101 of plasma RNA equivalents (purified RNA from volume of plasma). In some cases, the samples for analysis can contain between 0.5 μl and 5 μl of plasma RNA equivalents (purified RNA from volume of plasma). In some cases, the samples for analysis can contain about 0.1 μl, 0.5 μl, 1 μl, 2 μl, 3 μl, 4 μl, 5 μl, 6 μl, 7 μl, 8 μl, 9 μl or 10 μl of plasma RNA equivalents (purified RNA from volume of plasma).

The methods of the present invention can also include methods for altering the treatment regimen of a therapeutic intervention. Such methods comprise detecting the level of at least one miRNA, such as one or more miRNAs listed in any one of Tables 2-4, in a biological sample from a patient receiving the therapeutic intervention and altering the treatment regimen based on an increase or decrease in the level of the at least one miRNA in said biological sample. In some embodiments, the method comprises detecting two, three, four, five, ten, or more miRNAs (e.g., including all miRNAs) listed in one or more of Table 2-4. In some such embodiments, the miRNAs are detected using a customized detection platform, and thus less than 100, less than 50, or less than 25 miRNAs are detected, including the miRNAs from Table 2 to 4. Altering the treatment regimen can include but is not limited to changing and/or modifying the type of therapeutic intervention, the dosage at which the therapeutic intervention is administered, the frequency of administration of the therapeutic intervention, the route of administration of the therapeutic intervention, as well as any other parameters that would be well known by a physician to change and/or modify. For example, where miRNAs of Table 2 decrease during therapy or match reference levels, and or where miRNAs of Table 3 increase during therapy or match reference levels, the therapeutic intervention is continued. In embodiments where miRNAs of Table 2 do not decrease during therapy or match reference levels, and/or where miRNAs of Table do not increase during therapy or match reference levels, the therapeutic intervention is altered.

In certain embodiments, a panel of miRNAs is determined from one of Tables 1-4, and the panel classified on the basis of a classifier algorithm. For example, samples may be classified on the basis of threshold values as described, or based upon Mean and/or Median miRNA levels in one population or versus another (e.g., a population of healthy controls and population of patients with heart failure, or levels based on effective versus ineffective therapy).

Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

In some embodiments, the information regarding the increase or decrease in the level of at least one miRNA can be used to determine the treatment efficacy of treatment with the therapeutic intervention, as well as to tailor the treatment regimens of therapeutic interventions. In some embodiments the treatment efficacy can be used to determine whether to continue a therapeutic intervention. In some embodiments the treatment efficacy can be used to determine whether to discontinue a therapeutic intervention. In some embodiments the treatment efficacy can be used to determine whether to modify a therapeutic intervention. In some embodiments the treatment efficacy can be used to determine whether to increase or decrease the dosage of a therapeutic intervention. In some embodiments the treatment efficacy can be used to determine whether to change the dosing frequency of a therapeutic intervention. In some embodiments, the treatment efficacy can be used to determine whether to change the number or the frequency of administration of the therapeutic intervention. In some embodiments, the treatment efficacy can be used to determine whether to change the number of doses per day, per week, times per day. In some embodiments the treatment efficacy can be used to determine whether to change the dosage amount.

The methods of the present invention also provide methods for providing useful information for evaluating or monitoring the efficacy of a therapeutic intervention for treating a cardiac disorder. These methods can include determining the level of at least one miRNA, such as one or more miRNAs listed in any one of Tables 2-4, in a biological sample of a patient and providing the level of the at least one miRNA to an entity that provides a determination of the efficacy based on an increase or decrease in the level of the at least one miRNA.

The phrase “indicative of the therapeutic efficacy” and variants thereof can include any methods for determining that a therapeutic intervention is providing a benefit to a patient. The terms “therapeutic efficacy” and variants thereof are generally indicated by alleviation of one or more signs or symptoms associated with a cardiac disorder and alleviation of one or more signs or symptoms of the cardiac disorder being treated can be readily determined by one skilled in the art. “Therapeutic efficacy” may also refer to the prevention or amelioration of signs and symptoms of toxicities typically associated with standard therapeutic interventions for cardiac disorders. Methods for determining therapeutic efficacy may be specific to the cardiac disorder being treated and can include any methods well known in the art for determining that a treatment is providing a beneficial effect to a cardiac disorder patient. For example, evidence of therapeutic efficacy can include but is not limited to improvement or alleviation of one or more symptoms of cardiac hypertrophy, heart failure, or myocardial infarction in the subject, or in the delay in the transition from cardiac hypertrophy to heart failure. The one or more improved or alleviated symptoms can include, for example, increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased cardiac fibrosis, decreased collagen deposition in cardiac muscle, decreased left and right ventricular wall stress, decreased wall tension, increased quality of life, and decreased disease related morbidity or mortality. Further, therapeutic efficacy can also include general improvements in the overall health of the patient, such as but not limited to enhancement of patient life quality, increase in predicted survival rate, decrease in depression or decrease in rate of recurrence of the indication. (See, e.g., Physicians' Desk Reference (2010).)

Efficacy of a therapeutic intervention can also include evaluating or monitoring for the improvement of one or more symptoms of cardiac hypertrophy, heart failure, or myocardial infarction in the subject, or for the delay in the transition from cardiac hypertrophy to heart failure. The one or more improved symptoms may include, for example, increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased cardiac fibrosis, decreased collagen deposition in cardiac muscle, decreased left and right ventricular wall stress, decreased wall tension, increased quality of life and decreased disease related morbidity or mortality. In some embodiments, the measured levels of plasma miRNAs (e.g., any of the miRNAs listed in Tables 2-4) serve as a surrogate marker for efficacy of the therapeutic intervention.

Any of the compositions described herein may be comprised in a kit. In one embodiment, the kit contains a reagent for measuring at least one miRNA selected from any one of Tables 2-4 in a biological sample, instructions for measuring the at least one miRNA and instructions for evaluating or monitoring the efficacy of a therapeutic intervention for treating a cardiac disorder in a patient based on the level of the at least one miRNA. In some embodiments, the kit contains reagents for measuring the level of at least two, three, four, five, ten, or twenty miRNAs (or more), from one of Tables 2 or 3, or collectively from Tables 2 and 3. In some embodiments, the kit is customized for determining the efficacy of therapy for heart failure, and thus provides the reagents for determining 50 or less, 40 or less, 30 or less, or twenty five or fewer miRNAs, including the miRNAs of Tables 2 and 3.

In some embodiments, the kit contains a reagent for measuring one or more miRNAs selected from hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-126, hsa-miR-133a, hsa-miR-499, hsa-miR-19b, hsa-miR-199b-5p, hsa-miR-204, hsa-miR-145, hsa-miR-195, hsa-miR-125a-5p, hsa-miR-143, and hsa-miR-214, instructions for measuring one or more of these miRNAs, and instructions for evaluating or monitoring the efficacy of a therapeutic intervention for treating a cardiac disorder in a patient based on the level of one or more of these miRNAs. In particular embodiments, the kit contains a reagent for measuring one or more miRNAs selected from miR-423-5p, miR-106b, miR-16, miR-92a, miR-378*, miR-210, miR-378*, miR-20b, miR-93, miR-19b, miR-223, miR-21, miR-150, instructions for measuring one or more of these miRNAs, and instructions for evaluating or monitoring the efficacy of a therapeutic intervention for treating a cardiac disorder in a patient based on the level of one or more of these miRNAs. In some embodiments, the kit contains an miRNA-specific primer for reverse transcribing or amplifying a miRNA selected from hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-a26, hsa-miR-133a, hsa-miR-499, hsa-miR-19b, hsa-miR-199b-5p, hsa-miR-204, hsa-miR-145, hsa-miR-195, hsa-miR-125a-5p, hsa-miR-143, hsa-miR-214, and combinations thereof.

In some embodiments, the kit contains a reagent for measuring one or more miRNAs in a biological sample that are indicative of the therapeutic efficacy of a miRNA-based therapeutic. For instance, in one embodiment, the kit contains a reagent for measuring one or more miRNAs selected from miR-19b, miR-223, miR-423-5p, miR-106b, miR-16, miR-92a, miR-378, miR-210, miR-378*, miR-20b, and miR-93 in a biological sample, instructions for measuring one or more of these miRNAs, and instructions for evaluating or monitoring the efficacy of a chemically-modified antisense oligonucleotide targeting miR-208a and/or miR-208b for treating a cardiac disorder in a patient based on the levels of one or more of these miRNAs. In some embodiments, the kit contains reagents for measuring the level of at least two, three, four five, ten, or twenty miRNAs (or more), from one of Tables 2 or 3, or collectively from Tables 2 and 3. In some embodiments, the kit is customized for determining the efficacy of therapy for heart failure, and thus provides the reagents for determining 50 or less, 40 or less, 30 or less, or twenty five or fewer miRNAs, including the miRNAs of Tables 2 and 3.

In other embodiments, the kit contains a reagent for measuring one or more miRNAs in a biological sample that are indicative of the therapeutic efficacy of a standard of care therapeutic, such as an angiotensin-converting enzyme (ACE) inhibitor. For instance, in one embodiment, the kit contains a reagent for measuring one or more miRNAs selected from miR-150, miR-21, miR-223, miR-423-5p, miR-106b, miR-16, miR-92a, miR-378, miR-210, miR-378*, miR-20b, and miR-93 in a biological sample, instructions for measuring one or more of these miRNAs, and instructions for evaluating or monitoring the efficacy of an ACE inhibitor for treating a cardiac disorder in a patient based on the levels of one or more of these miRNAs. In some embodiments, the kit contains reagents for measuring the level of at least two, three, four five, ten, or twenty miRNAs (or more), from one of Tables 2 or 3, or collectively from Tables 2 and 3. In some embodiments, the kit is customized for determining the efficacy of therapy for heart failure, and thus provides the reagents for determining 50 or less, 40 or less, 30 or less, or twenty five or fewer miRNAs, including the miRNAs of Tables 2 and 3.

In some embodiments, the kit can further contain one or more normalization controls. In some other embodiments, the one or more normalization controls are provided as one or more separate reagents for spiking samples or reactions. The normalization control can be added in a range of from about 0.1 fmol to about 5 mol. In some embodiments, the normalization control is added at about 0.1 fmol, 0.5 fmol, 1 fmol, 2 fmol, 3 fmol, 4 fmol or 5 fmol. In some embodiments, the at least one normalization control is a non-endogenous RNA or miRNA, or a miRNA not expressed in the sample. In some embodiments, the at least one normalization control is a C. elegans miRNA. In some embodiments, the at least one normalization control is cel-miR-2, cel-lin-4 or ath-miR-159a. In some embodiments, the C. elegans sequences used were cel-miR-2 (UAUCACAGCCAGCUUUGAUGUGC, SEQ ID NO:5), and cel-lin-4 (UCCCUGAGACCUCAAGUGUGUGA; SEQ ID NO:6). The sequence for ath-miR159a used was (UUUGGAUUGAAGGGAGCUCUA, SEQ ID NO: 7).

In some embodiments, the kit can further contain a TaqMan probe specific for each miRNA of the kit. In some embodiments, the TaqMan probe is specific for a miRNA selected from the group consisting of hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-126, hsa-miR-133a, hsa-miR-499, hsa-miR-19b, hsa-miR-199b-5p, hsa-miR-204, hsa-miR-145, hsa-miR-195, hsa-miR-125a-5p, hsa-miR-143, and hsa-miR-214.

In some embodiments, the kit is contemplated for use with a biological sample from a patient receiving treatment for a cardiac disorder. In further embodiments, the biological sample is plasma or serum obtained from a patient receiving treatment for a cardiac disorder. In yet further embodiments, the cardiac disorder is myocardial infarction, pathologic cardiac hypertrophy, heart failure or hypertension.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

Such kits may also include components that preserve or maintain the reagents or that protect against their degradation. Such components may be DNAse-free, RNAse-free or protect against nucleases (e.g., RNAses and DNAses). Such kits generally will comprise, in suitable means, distinct containers for each individual reagent or solution.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used for the manipulation or characterization of miRNA.

The therapeutic interventions of the present invention can include any combination of therapies used to treat cardiac disorders. Examples can include but are not limited to miRNA based therapeutics (including antisense oligonucleotides), antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. (See, e.g., U.S. Patent Application No. 2010/0317713; incorporated by reference herein in its entirety.)

In some embodiments, the therapeutic intervention is a miRNA based therapeutic. In some embodiments, the miRNA based therapeutic is an antisense oligonucleotide. The antisense oligonucleotides may be ribonucleotides or deoxyribonucleotides. In some embodiments, the miRNA based therapeutic is an antisense oligonucleotide targeting a miRNA expressed in heart tissue. In some embodiments, the therapeutic intervention is an antisense oligonucleotide targeting miR-208a and/or miR-208b. In certain such embodiments, a change in the measured level of the miRNA relative to the level of the miRNA in the control sample or pre-determined reference value is indicative of decreased expression of miR-208a and/or miR-208b in heart tissue. In such embodiments, the level of one or more miRNAs selected from miR-19b, mR-223, miR-423-5p, miR-106b, miR-16, miR-92a, miR-378, miR-210, miR-378*, miR-20b, and miR-93 as compared to the level of said one or more miRNAs in a control sample or pre-determined reference value is indicative of the therapeutic efficacy of a chemically-modified antisense oligonucleotide targeting miR-208a and/or miR-208b. In one particular embodiment, the level of miR-19b and/or miR-223 as compared to the level of miR-19b and/or miR-223 in a control sample or pre-determined reference value is indicative of the therapeutic efficacy of a chemically-modified antisense oligonucleotide targeting miR-208a and/or miR-208b. In certain embodiments, an increase in the level of miR-19b as compared to the level of miR-19b in a control sample or pre-determined reference value is indicative of decreased expression of miR-208a and/or miR-208b in heart tissue. In other embodiments, a decrease in the level of miR-223 as compared to the level of miR-223 in a control sample or pre-determined reference value is indicative of decreased expression of miR-208a and/or miR-208b in heart tissue.

Preferably, the antisense oligonucleotide therapeutics have at least one chemical modification (i.e., the oligonucleotide is chemically modified). For instance, suitable antisense oligonucleotides may be comprised of one or more “conformationally constrained” or bicyclic sugar nucleoside modifications, for example, “locked nucleic acids.” in some embodiments, the miRNA based therapeutic is a chemically-modified antisense oligonucleotide. In some embodiments, the miRNA based therapeutic is a chemically-modified antisense oligonucleotide targeting a miRNA expressed in heart tissue. In some embodiments, the chemically-modified antisense oligonucleotide targets miR-208a and/or miR-208b.

The miR-208a and miR-208b microRNAs have been described in WO 2008/016924, WO 2009/018492 and WO 2010/091204. The pre-miRNA encoding sequences for miR-208a for human is shown below as SEQ ID NO: 8 and the mature miR-208a sequence is provided in SEQ ID NO: 9. (See, e.g., PCT/LUS2010/023234, which is incorporated herein by reference in its entirety.) The pre-miRNA encoding sequences for miR-208b for human is shown below as SEQ ID NO: 10 and the mature miR-208b sequence is provided in SEQ ID NO: 11.

Human pre-miR-208a
(SEQ ID NO: 8)
ACGGGCGAGC TTTTGGCCCG GGTTATACCT GATGCTCACG
TATAAGACGA GCAAAAAGCT TGTTGGTCAG A
Mature miR-208a
(SEQ ID NO: 9)
AUAAGACGAGCAAAAAGCUUGU
Human pre-miR-208b
(SEQ ID NO: 10)
CCUCUCAGGGAAGCUUUUUGCUCGAAUUAUGUUUCUGAUCCGAAUAUAAG
ACGAACAAAAGGUUUGUCUGAGGGCAG
Mature miR-208b
(SEQ ID NO: 11)
AUAAGACGAACAAAAGGUUUGU

The antisense oligonucleotides targeting miR-208a/miR-208b can contain combinations of LNAs or other modified nucleotides and ribonucleotides or deoxyribonucleotides. Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). For instance, antisense oligonucleotides, particularly those of shorter lengths (e.g., less than 15 nucleotides) can comprise one or more affinity enhancing modifications, such as, but not limited to, LNAs, bicyclic nucleosides, phosphonoformates, 2′ O alkyl and the like.

In some embodiments, suitable antisense oligonucleotides are 2′-O-methoxyethyl S “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Preferable antisense oligonucleotides useful for inhibiting the activity of miRNAs are about 5 to about 50 nucleotides in length, about 10 to about 30 nucleotides in length, or about 20 to about 25 nucleotides in length. In certain embodiments, antisense oligonucleotides targeting miR-208a and/or miR-208b are about 8 to about 18 nucleotides in length, and in other embodiments about 12 to 16 nucleotides in length. In particular, any 8-mer or longer that is complementary to miR-208a or miR-208b may be used, i.e., any antimir sequence that is complementary to any consecutive sequence in miR-208a or miR-208b, starting from the 5′ end of the miR to the 3′ end of the mature sequence. Antisense oligonucleotides may in some cases comprise a sequence that is at least partially complementary to a mature miRNA sequence, e.g., at least about 75%, 80%, 85%, 90%, 95%, 96%, 97%, 98%, or 99% complementary to a mature miRNA sequence. In some embodiments, the antisense oligonucleotide may be substantially complementary to a mature miRNA sequence, that is at least about 95%, 96%, 97%, 98%, or 99% complementary to a target polynucleotide sequence. In one embodiment, the antisense oligonucleotide comprises a sequence that is 100% complementary to a mature miRNA sequence.

“Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. LNAs are described, for example, in U.S. Pat. No. 6,268,490, U.S. Pat. No. 6,316,198, U.S. Pat. No. 6,403,566, U.S. Pat. No. 6,770,748, U.S. Pat. No. 6,998,484, U.S. Pat. No. 6,670,461, and U.S. Pat. No. 7,034,133, all of which are hereby incorporated by reference in their entireties. LNAs are modified nucleotides or ribonucleotides that contain an extra bridge between the 2′ and 4′ carbons of the ribose sugar moiety resulting in a “locked” conformation. Other suitable locked nucleotides that can be incorporated in the oligonucleotides of the invention include those described in U.S. Pat. No. 6,833,361, which is hereby incorporated by reference in their entirety.

In other embodiments, the antisense oligonucleotides are antagomirs. “Antagomirs” are single-stranded, chemically-modified ribonucleotides that are at least partially complementary to the miRNA sequence. Antagomirs may comprise one or more modified nucleotides, such as 2′-O-methyl-sugar modifications. In some embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a steroid such as cholesterol, a fatty acid, a vitamin, a carbohydrate, a peptide or another small molecule ligand at its 3′ end. Antagomirs suitable for inhibiting miRNAs may be about 15 to about 50 nucleotides in length, more preferably about 18 to about 30 nucleotides in length, and most preferably about 20 to about 25 nucleotides in length. “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to the mature miRNA sequence.

The therapeutic intervention also includes other standard cardiac therapies, in addition to microRNA targeted treatments as described above. In these embodiments, the therapeutic interventions of the present invention can include antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. See, e.g., U.S. Patent Application No. 2010/0317713, incorporated by reference herein in its entirety.

In one embodiment, the present invention provides a method for evaluating or monitoring the efficacy of a standard of care cardiac therapy, such as an ACE inhibitor, by measuring the level of at least one miRNA in a biological sample. In some embodiments, the method comprises measuring the level of at least one miRNA selected from miR-150, miR-21, miR-223, miR-423-5p, miR-106b, miR-16, miR-92a, miR-378, miR-210, miR-378*, miR-20b, and miR-93 in a biological sample from a patient receiving the ACE inhibitor; and comparing the measured level to the level of said at least one miRNA in a control sample or pre-determined reference value, wherein the measured level of said at least one miRNA is indicative of the therapeutic efficacy of the ACE inhibitor. In another embodiment, the method comprises measuring the level of miR-21 and/or miR-150 in a biological sample from a patient receiving the ACE inhibitor; and comparing the measured level to the level of miR-21 and/or miR-150 in a control sample or pre-determined reference value, wherein the measured level of said at least one miRNA is indicative of the therapeutic efficacy of the ACE inhibitor.

In certain embodiments, the measured level of one or more miRNAs relative to the level of said one or more miRNAs in a control sample or pre-determined reference value is indicative of the therapeutic efficacy of a combination therapy. The combination therapy can two or more miRNA-based therapeutics, two or more standard of care therapies, or a combination of a miRNA-based therapeutic and a standard of care therapy. For instance, the combination therapy may be a chemically-modified antisense oligonucleotide targeting a miRNA expressed in the heart and a standard of care therapy, such as an ACE inhibitor. In one particular embodiment, the combination therapy is a chemically-modified antisense oligonucleotide targeting miR-208a and/or miR-208b and an ACE inhibitor, such as captopril. In such embodiments, the level of one or more miRNAs selected from miR-21, miR-150, miR-378, miR-378*, and miR-93 relative to the level of one or more of these miRNAs in a control sample or pre-determined reference value is indicative of the therapeutic efficacy of a combination therapy of an antimiR-208 oligonucleotide and an ACE inhibitor (e.g. captopril). In some embodiments, the level of miR-378 and/or miR-378* relative to the level of miR-378 and/or miR-378* in a control sample or pre-determined reference value is indicative of the therapeutic efficacy of a combination therapy of an antimiR-208 oligonucleotide and an ACE inhibitor (e.g. captopril).

In still other aspects, the invention provides a method for treating a patient for heart failure, the patient having elevated levels (e.g., serum or plasma levels) of one or more of miR-223, miR-16, miR-93, miR-106(b), and/or miR-423-5p, or other combination of up-regulated or down-regulated markers (e.g., combination of at least two) listed in Table 1. In accordance with this aspect, the patient is treated for heart failure with conventional therapy (such as an ACE inhibitor), or with an anti-miR therapeutic strategy described herein. In some embodiments, the therapy is with an anti-miR-208(a) antisense inhibitor or an anti-miR-208(b) antisense inhibitor. The levels of miR-223, miR-16, miR-93, miR-106(b) and/or miR-423-5p may be determined prior to treatment, and/or during treatment to monitor efficacy of the regimen. In some embodiments, the treatment is an antisense anti-miR-208(b) inhibitor, and the level of miR-19(b) or a combination of markers listed in one or more of Tables 2-4 is monitored during treatment. In some embodiments, the anti-miR-208(b) therapy is as described herein or in U.S. Provisional Application No. 61/495,224, which is hereby incorporated by reference in its entirety. For example, the inhibitor may be an antisense inhibitor of from 10 to 18 nucleotides (e.g., 16 nucleotides), and/or containing nine locked nucleic acids, in addition to other chemical motifs. The inhibitor may be M-10101 as described in U.S. Provisional Application No. 61/495,224, and may be administered subcutaneously.

In some embodiments, the therapeutic intervention includes administration of an agent that lowers the concentration of one of more blood lipids and/or lipoproteins, known herein as an “antihyperlipoproteinemic,” can be part of the therapeutic interventions according to the present invention.

Examples of antihyperlipoproteinemics can include but are not limited to acifran, azacosterol, benfluorex, p-benzalbutyramide, carnitine, chondroitin sulfate, clomestrone, detaxtran, dextran sulfate sodium, 5, 8, 11, 14, 17-eicosapentaenoic acid, eritadenine, furazabol, meglutol, melinamide, mytatrienediol, ornithine, y-oryzanol, pantethine, pentaerythritol tetraacetate, α-phenylbutyramide, pirozadil, probucol (lorelco), p-sitosterol, sultosilic acid-piperazine salt, tiadenol, triparanol and xenbucin.

In some embodiments, antihyperlipoproteinemic agents can further comprise an aryloxyalkanoicifibric acid derivative, a resin/bile acid sequesterant, a HMG CoA reductase inhibitor, a nicotinic acid derivative, a thyroid hormone or thyroid hormone analog, a miscellaneous agent or a combination thereof.

Examples of aryloxyalkanoic/fibric acid derivatives can include but are not limited to beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

Examples of resins/bile acid sequesterants can include but are not limited to cholestyramine (cholybar, questran), colestipol (colestid) and polidexide.

Examples of 1-1MG CoA reductase inhibitors can include but are not limited to lovastatin (mevacor), pravastatin (pravochol) and simvastatin (zocor).

Examples of nicotinic acid derivatives can include but are not limited to nicotinate, aceplmox, niceritrol, nicoclonate, niconol and oxiniacic acid.

Examples of thyroid hormones and analogs thereof can include but are not limited to etoroxate, thyropropic acid and thyroxine.

Examples of an antiarteriosclerotic can include but are not limited to pyridinol carbamate.

In some embodiments, administration of an agent that aids in the removal or prevention of blood clots may be combined with administration of a modulator, particularly in treatment of athersclerosis and vasculature (e.g., arterial) blockages. Examples of antithrombotic and/or fibrinolytic agents can include but are not limited to anticoagulants, anticoagulant antagonists, antiplatelet agents, thrombolytic agents, throinbolytic agent antagonists or combinations thereof.

In some embodiments, antithrombotic agents that can be included are those that are administered orally, such as, for example, aspirin and warfarin (coumadin).

Anticoagulants can include but are not limited to acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodiuim, oxazidione, pentosan polysulfate, phenindione, phenprocoumon, phosvitin, picotamide, tioclomarol and warfarin.

Antiplatelet agents can include but are not limited to aspirin, a dextran, dipyridamole (persantin), heparin, sulfinpyranone (anturane) and ticlopidine (ticlid).

Thrombolytic agents can include but are not limited to tissue plasminogen activator (activase), plasmin, pro-urokinase, urokinase (abbokinase) streptokinase (streptase) and anistreplasel APSAC (eminase).

In other embodiments, wherein a subject is suffering from a hemorrhage or an increased likelihood of hemorrhaging, an agent that may enhance blood coagulation can also be employed. Examples of blood coagulation promoting agents include thrombolytic agent antagonists and anticoagulant antagonists.

Thrombolytic agent antagonists can include but are not limited to amiocaproic acid (amicar) and tranexamic acid (amstat).

Anticoagulant antagonists can include but are not limited to protamine and vitamin K.

In some embodiments, the therapeutic intervention is an antithrombotic/fibrinolytic agent. Antithrombotic fibrinolytic agents can include but are not limited to anagrelide, argatroban, cilstazol, daltroban, defibrotide, enoxaparin, fraxiparine, indobufen, lamoparan, ozagrel, picotamide, plafibride, tedelparin, ticlopidine and triflusal.

In some embodiments, the therapeutic intervention is an antiarrhythmic. Antiarrhythmic agents can include but are not limited to Class I antiarrhythmic agents (sodium channel blockers), Class II antiarrhythmic agents (beta-adrenergic blockers), Class III antiarrhythmic agents (repolarization prolonging drugs), Class IV antiarrhythmic agents (calcium channel blockers) and miscellaneous antiarrhythric agents.

Examples of sodium channel blockers can include but are not limited to Class IA, Class IB and Class IC antiarrhythmic agents. Non-limiting examples of Class IA antiarrhythmic agents include disppyramide (norpace), procainamide (pronestyl) and quinidine (quinidex). Examples of Class IB antiarrhythmic agents can include but are not limited to lidocaine (xylocalne), tocamide (tonocard) and mexiletine (mexitil). Examples of Class IC antiarrhythmic agents can include but are not limited to encamide (enkaid) and flecamide (tambocor).

Examples of a beta blocker, otherwise known as a p-adrenergic blocker, a p-adrenergic antagonist or a Class II antiarrhythmic agent, can include but are not limited to acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol.

In some embodiments, the beta blocker can comprise an aryloxypropanolamine derivative. Examples of aryloxypropanolamine derivatives can include but are not limited to acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, mrnoprolol, nadolol, nipradilol, oxprenolol, penbutolol, pindolol, propanolol, talinolol, tertatolol, tinolol and toliprolol.

Examples of agents that prolong repolarization, also known as a Class III antiarrhythmic agent, can include but are not limited to include amiodarone (cordarone) and sotalol (betapace).

Examples of a calcium channel blocker, otherwise known as a Class IV antiarrhythmic agent, can include but are not limited to an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In some embodiments, a calcium channel blocker comprises a long-acting dihydropyridine (nifedipine-type) calcium antagonist.

Examples of miscellaneous antiarrhythmic agents can include but are not limited to include adenosine (adenocard), digoxin (lanoxin), acecamide, ajmaline, amoproxan, aprindine, bretylium tosylate, bunaftine, butobendine, capobenic acid, cifenline, disopyranide, hydro quinidine, indecamide, ipatropium bromide, lidocaine, lorajmine, lorcamide meobentine, moricizine, pirmenol, prajmaline, propafenone, pyrinoline, quinidine polygalacturonate, quinidine sulfate and viquidil.

In some embodiments, the therapeutic intervention is an antihypertensive agent. Examples of antihypertensive agents can include but are not limited to sympatholytic, alpha/beta blockers, alpha blockers, anti-angiotensin II agents, beta blockers, calcium channel blockers, vasodilators and miscellaneous antihypertensives.

Examples of an alpha blocker, also known as an c-adrenergic blocker or an o-adrenergic antagonist, can include but are not limited to amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Quinazoline derivatives can include but are not limited to alfuzosin, bunazosin, doxazosin, prazosin, terazosin and trimazosin.

In certain embodiments, an antihypertensive agent is both an alpha and beta adrenergic antagonist. Examples of an alpha/beta blocker can include but are not limited to labetalol (normodyne, trandate).

Examples of anti-angiotensin II agents can include but are not limited to angiotensin converting enzyme inhibitors and angiotensin II receptor antagonists. Angiotensin converting enzyme inhibitors (ACE inhibitors) can include but are not limited to alacepril, enalapril (vasotec), captopril, cilazapril, delapril, enalaprilat, fosinopril, lisinopril, moveltopril, perindopril, quinapril and ramipril. Examples of an angiotensin II receptor blocker, also known as an angiotensin II receptor antagonist, an ANG receptor blocker or an ANG-II type-I receptor blocker (ARBS), include but are not limited to angiocandesartan, eprosartan, irbesartan, losartan and valsartan.

Examples of a sympatholytic include a centrally acting sympatholytic or a peripherially acting sympatholytic. Examples of a centrally acting sympatholytic, also known as an central nervous system (CNS) sympatholytic, can include but are not limited to clonidine (catapres), guanabenz (wytensin) guanfacine (tenex) and methyldopa (aldomet).

Examples of a peripherally acting sympatholytic can include but are not limited to a ganglion blocking agent, an adrenergic neuron blocking agent, β-adrenergic blocking agent or a alpha1-adrenergic blocking agent. Examples of a ganglion blocking agent include mecarnylamine (inversine) and trimethaphan (arfonad). Examples of an adrenergic neuron blocking agent can include but are not limited to guanethidine (ismelin) and reserpine (serpasil).

Examples of a β-adrenergic blocker can include but are not limited to acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren).

Examples of alpha1-adrenergic blocker can include but are not limited to prazosin (minipress), doxazocin (cardura) and terazosin (hytrin).

In some embodiments, the therapeutic intervention can comprise a vasodilator (e.g., a cerebral vasodilator, a coronary vasodilator or a peripheral vasodilator). In other embodiments, a vasodilator comprises a coronary vasodilator. Examples of a coronary vasodilator include but are not limited to amotriphene, bendazol, benfurodil hemisuccinate, benziodarone, chlioracizine, chromonar, clobenfurol, clonitrate, dilazep, dipyridamole, droprenilamine, efloxate, erythrityl tetranitrane, etafenone, fendiline, floredil, ganglefene, herestrol bis(p-dinoeylaminoethyl ether), hexobendine, itramin tosylate, khellin, lidoflanine, mannitol hexanitrane, medibazine, nicorglycerin, pentaerythritol tetranitrate, pentrinitrol, perhexyline, pimefyiline, trapidil, tricromyl, trimetazidine, troInitrate phosphate and visnadine.

In some embodiments, a vasodilator can comprise a chronic therapy vasodilator or a hypertensive emergency vasodilator. Examples of a chronic therapy vasodilator can include but are not limited to hydralazine (apresoline) and minoxidil (loniten). Examples of a hypertensive emergency vasodilator can include but are not limited to nitroprusside (nipride), diazoxide (hyperstat IV), hydralazine (apresoline), minoxidil (loniten) and verapamil.

In some embodiments, the therapeutic intervention can include an antihypertensive. Examples of miscellaneous antihypertensives include but are not limited to ajmaline, γ-amino butyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzo limine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain embodiments, an antihypertensive can comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quaternary ammoniam compound, a reserpine derivative or a suflonamide derivative.

Examples of arylethanolamine derivatives can include but are not limited to amosulalol, bufuralol, dilevalol, labetalol, pronethalol, sotalol and sulfinalol.

Examples of benzothiadiazine derivatives can include but are not limited to althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

Examples of N-carboxyalkyl(peptide/lactam) derivatives can include but are not limited to alacepril, captopril, cilazapril, delapril, enalapril, enalaprilat, fosinopril, lisinopril, moveltipril, perindopril, quinapril and ramipril.

Examples of dihydropyridine derivatives can include but are not limited to amlodipine, felodipine, isradipine, nicardipine, nifedipine, nilvadipine, nisoldipine and nitrendipine.

Examples of guanidine derivatives can include but are not limited to bethanidine, debrisoquin, guanabenz, guanacline, guanadrel, guanazodine, guanethidine, guanfacine, guanochlor, guanoxabenz and guanoxan.

Examples of hydrazines/phthalazines can include but are not limited to budralazine, cadralazine, dihydralazine, endralazine, hydracarbazine, hydralazine, pheniprazine, pildralazine and todralazine.

Examples of imidazole derivatives can include but are not limited to clonidine, lofexidine, phentolamine, tiamenidine and tolonidine.

Examples of quaternary ammonium compounds can include but are not limited to azamethonium bromide, chlorisondamine chloride, hexamethonium, pentacynium bis(methylsulfate), pentamethoniumi bromide, pentolinium tartrate, phenactropiniutm chloride and trimethidinium methosulfate.

Examples of reserpine derivatives can include but are not limited to bietaserpine, deserpidine, rescinnamine, reserpine and syrosingopine.

Examples of sulfonamide derivatives can include but are not limited to ambuside, clopamide, furosemide, indapamide, quinethazone, trip amide and xipamide.

Examples of agents for the treatment of congestive heart failure can include but are not limited to anti-angiotensin II agents, afterload-preload reduction treatment, diuretics and inotropic agents.

Examples of a diuretic can include but are not limited to a thiazide or benzothiadiazine derivative (e.g., althiazide, bendroflumethazide, benzthiazide, benzylhydrochiorchlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamnphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenami de, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, trip amide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzo limine, perhexyline, ticmafen and urea.

Examples of a positive inotropic agent, also known as a cardiotonic, can include but are not limited to acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol. in some embodiments. an intropic agent is a cardiac glycoside, a beta-adrenergic agonist or a phosphodiesterase inhibitor. Examples of a cardiac glycoside can include but are not limited to digoxin (lanoxin) and digitoxin (crystodigin). Examples of a β-adrenergic agonist include but are not limited to albuterol, bambuterol, bitolterol, carbuterol, clenbuterol, clorprenaline, denop amine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Examples of a phosphodiesterase inhibitor can include but are not limited to aminone (inocor).

Antianginal agents may comprise organonitrates, calcium channel blockers, beta blockers and combinations thereof. Examples of organonitrates, also known as nitrovasodilators, can include but are not limited to nitroglycerin (nitro-bid, nitrostat), isosorbide dinitrate (isordil, sorbitrate) and amyl nitrate (aspirol, vaporole).

Endothelin (ET) is a 21-amino acid peptide that has potent physiologic and pathophysiologic effects that appear to be involved in the development of heart failure. The effects of ET are mediated through interaction with two classes of cell surface receptors. Inhibiting the ability of ET to stimulate cells involves the use of agents that block the interaction of ET with its receptors. Examples of endothelin receptor antagonists (ERA) can include but are not limited to Bosentan, Enrasentan, Ambrisentan, Darusentan, Tezosentan, Atrasentan, Avosentan, Clazosentan, Edonentan, sitaxsentan, TBC 3711, BQ 123, and BQ 788.

In certain embodiments, therapeutic intervention can include a secondary therapeutic aspect such as for example a surgery of some type, which includes, for example, preventative, diagnostic or staging, curative and palliative surgery. Surgery, and in particular a curative surgery, may be used in conjunction with other therapies, including one or more other agents as described herein. Such surgical therapeutic agents for vascular and cardiovascular diseases and disorders are well known to those of skill in the art, and may include, but are not limited to, performing surgery on an organism, providing a cardiovascular mechanical prostheses, angioplasty, coronary artery reperfision, catheter ablation, providing an implantable cardioverter defibrillator to the subject, mechanical circulatory support or a combination thereof. Examples of a mechanical circulatory support that may be used in the present invention comprise an intra-aortic balloon counterpuisation, left ventricular assist device or combination thereof.

The present invention also includes methods and kits for predicting or assessing the level of severity of heart failure or heart failure progression in a patient. In one embodiment, the present invention provides a method of predicting or assessing the level of severity of heart failure or heart failure progression in a patient comprising measuring the level of at least one miRNA selected from Table 1 in a biological sample from a patient; and comparing the measured level to the level of said at least one miRNA in a control sample (e.g. a sample obtained from a healthy, age-matched subject or a sample obtained from a subject not suffering from or diagnosed with heart failure) or pre-determined reference value, wherein the measured level of said at least one miRNA is indicative of the level of severity of heart failure or heart failure progression in the patient. The biological sample can be any sample described herein. In certain embodiments, plasma or serum samples are preferred.

In some embodiments, an increase in the measured level of the miRNA relative to the level of the miRNA in the control sample or pre-determined reference value is indicative of the level of severity of heart failure or heart failure progression in the patient. For instance, in such embodiments, when the level of one or more miRNAs selected from hsa-miR-16, hsa-miR-223, hsa-miR-320, hsa-miR-150, hsa-miR-378, hsa-miR-92a, hsa-miR-423-5p, hsa-miR-133a, hsa-miR-22, hsa-miR-21, hsa-miR-210, hsa-miR-20b, hsa-miR-499, hsa-miR-378*, hsa-miR-106b, hsa-miR-155, and hsa-miR-93 is increased when compared to the level in a control sample or pre-determined reference value, the increase is indicative of the level of severity of heart failure or heart failure progression in the patient.

In other embodiments, a reduction or decrease in the measured level of the miRNA relative to the level of the miRNA in the control sample (e.g., a sample obtained from a healthy, age-matched subject or a sample obtained from a subject not suffering from or diagnosed with heart failure) or pre-determined reference value is indicative of the level of severity of heart failure or heart failure progression in the patient. For instance, in such embodiments, when the level of one or more miRNAs selected from hsa-miR-204, hsa-miR-199b-5p, hsa-miR-125a-5p, hsa-miR-143, and hsa-miR-195 is decreased when compared to the level in a control sample or pre-determined reference value, the decrease is indicative of the level of severity of heart failure or heart failure progression in the patient.

The present invention also encompasses kits for predicting or assessing the level of severity of heart failure or heart failure progression in a patient. In one embodiment, the kit comprises a reagent for measuring at least one miRNA selected from Table 1 in a biological sample and instructions for measuring said at least one miRNA for predicting or assessing the level of severity of heart failure or heart failure progression in a patient. For instance, in certain embodiments, the kit comprises a miRNA-specific primer for reverse transcribing or amplifying a miRNA selected from of hsa-miR-204, hsa-miR-199b-5p, hsa-miR-125a-5p, hsa-miR-143, hsa-miR-195, hsa-miR-16, hsa-miR-223, hsa-miR-320, hsa-miR-150, hsa-miR-378, hsa-miR-92a, hsa-miR-423-5p, hsa-miR-133a, hsa-miR-22, hsa-miR-21 hsa-miR-210, hsa-miR-20b, hsa-miR-499, hsa-miR-378*, hsa-miR-106b, hsa-miR-155, and hsa-miR-93, and instructions for measuring said at least one miRNA for predicting or assessing the level of severity of heart failure or heart failure progression in a patient. The kits can further comprise additional reagents described herein for kits for evaluating or monitoring the therapeutic efficacy of cardiac treatments, such as normalization controls and Taqman probes specific for each of the miRNAs in the kit.

Cardiac disorders can include any disorders that affect the cardiovascular system, including the heart and/or blood vessels, such as arteries and veins. The cardiac disorders contemplated by the methods of the present invention can include cardiac diseases as well as cardiac failure. Cardiac disorders can also include disorders affecting the kidneys. Cardiac disorders can include but are not limited to heart failure, cardiac hypertrophy, coronary heart disease, cardiovascular disease, cardiomyopathy, ischaemic heart disease, hypertensive heart disease, inflammatoryl heart disease, valvular heart disease, atherosclerosis, cardiorenal disease, renal disease and other renal disorders. In some embodiments, the cardiac disorder is myocardial infarction, pathologic cardiac hypertrophy, heart failure or hypertension.

Cardiovascular diseases can include but are not limited to diabetes mellitus, hypertension, hyperhomocysteinemia and hypercholesterolemia.

Cardiomyopathies can include but are not limited to alcoholic cardiomyopathy, coronary artery disease, congenital heart disease, ischemic (or ischaemic) cardiomyopathy, hypertensive cardiomyopathy, valvular cardiomyopathy, inflammatory cardiomyopathy and myocardiodystrophy.

Hypertensive heart diseases can include but are not limited to left ventricular hypertrophy, coronary heart disease, heart failure (including congestive), hypertensive cardiomyopathy, cardiac arrhythmias and renal disorders.

Inflammatory heart diseases can include but are not limited to endocarditis, inflammatory cardiomegaly and myocarditis.

The following examples are included solely to illustrate various aspects of the invention. However, those of skill in the art should, in light of the present disclosure, appreciate that the invention is equally applicable to any human or other animal.

### EXAMPLES

### Example 1

### Blood-Borne miRNAs as Surrogate Markers of Drug Efficacy for Cardiac Conditions

The importance of miRNAs for cardiac function and dysfunction suggests opportunities for therapeutically exploiting the biology of miRNAs in the setting of heart disease. Single-stranded RNA oligonucleotides have been shown to be effective in inactivating miRNAs in vivo through complementary base pairing (Elmen, J. et al. (2008) Nature 452, 896-899; Elmen, J. et al. (2008) Nucleic Acids Res 36, 1153-1162; Krutzfeldt, J. et al. (2007) Nucleic Acids Res 35, 2885-2892; Krutzfeldt, J. et al. (2005) Nature 438, 685-689; Lanford, R. E. et al. (2010) Science 327, 198-201), and represent a potentially effective means of inactivating pathological miRNAs.

This example describes the therapeutic efficacy in a heart failure model of an antisense oligonucleotide targeting miR-208a. Specifically, we show that systemic delivery of locked nucleic acid (LNA)-modified antisense oligonucleotides against miR-208a is sufficient to induce specific, potent, and sustained silencing of miR-208a in the heart. Moreover, anti-miR-208a dose-dependently blunts stress-induced remodeling, functional deterioration, and cardiac myosin switching while improving general health and survival in a rat model of diastolic heart failure (Dahl salt-sensitive rats). Interestingly, we show that the physiological effects of antimiR-208a in hypertensive rats are mirrored by significant changes in plasma levels of circulating miRNAs, suggesting that these circulating miRNAs are surrogate markers of drug efficacy.

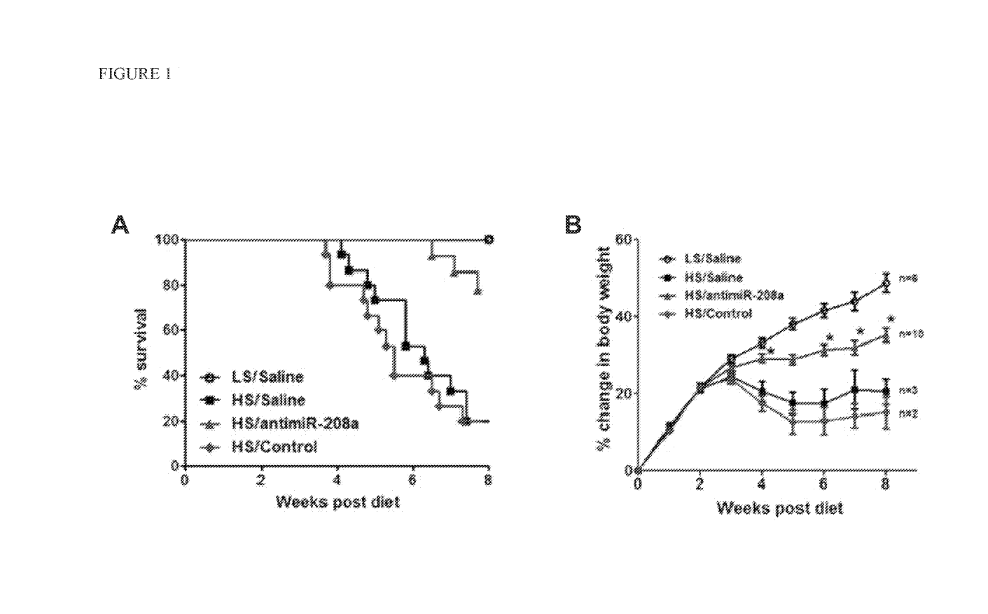

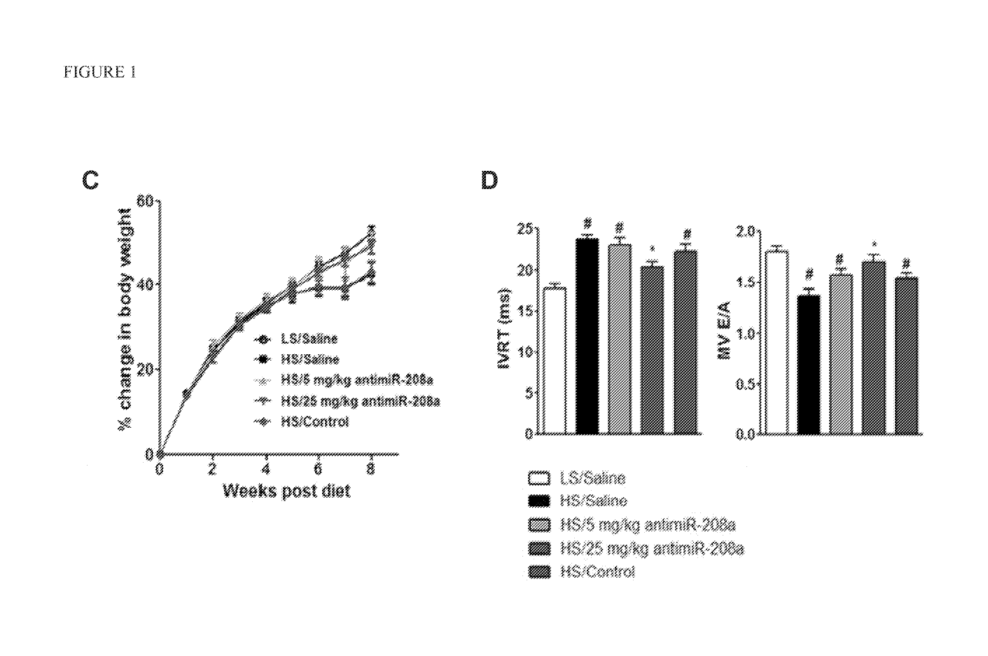

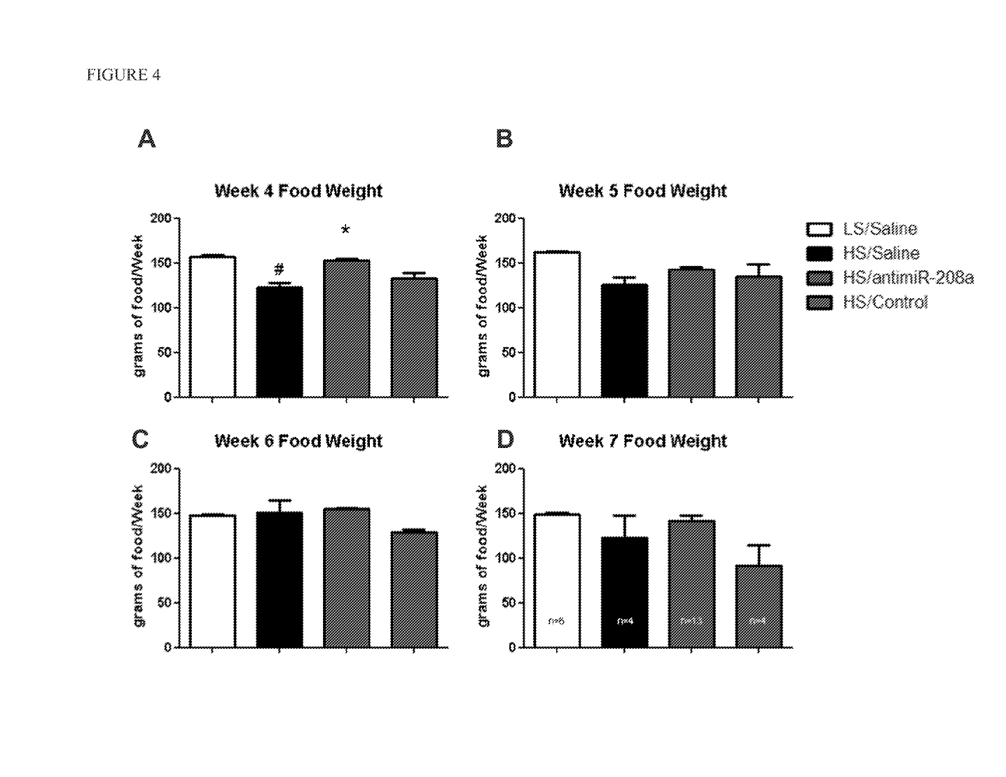

To determine the therapeutic potential of miR-208a inhibition in vivo, we designed an antimiR targeting bases 2 to 17 of the 5′ region of mature miR-208a and containing a combination of LNA and DNA linked by phosphorothioate bonds. The sequence of the antimiR-208a, which is referred to as M-10101, was 5′-CTTTTTGCTCGTCTTA-3′ (SEQ ID NO: 12). To test the therapeutic relevance of miR-208a inhibition, we used Dahl salt-sensitive rats that were either fed a low-salt (LS) diet (0.25% NaCl) or a high-salt (HS) diet (8.0% NaCl) starting at 8 weeks of age. Initiation of an HS diet in Dahl salt-sensitive rats results in chronic hypertension, which progresses to a model of congestive heart failure associated primarily with diastolic dysfunction (Rodenbaugh et al. (2007) Am J Physiol Heart Circ Physiol. 293, H1705-H1713). After one week on the HS diet, rats were administered saline, 25 mg/kg antimiR-208a, or 25 mg/kg scrambled control oligo subcutaneously every two weeks. After 3-4 weeks on the HS diet, the saline- and control-treated animals began to show signs of discomfort and died, whereas subcutaneous delivery of antimiR-208a significantly alleviated these symptoms (FIG. 1A). As an indication of health, we monitored body weight during the duration of the study. Dahl rats on the HS diet injected with either saline or the control oligo exhibited significant reductions in weight gain compared with controls on the LS diet. HS/antimiR-208a treated rats, however, showed comparable weight gain (FIG. 1B). To exclude the possibility antimiR-208a-treated animals were maintaining weight through ingesting less of the 8% HS diet, food intake was monitored, which showed a comparable ingestion between all HS-fed groups (FIG. 4).

To obtain additional insight into the basis for the protective effects seen in response to antimiR-208a, subsequent studies were done using a 4.0% NaCl diet, during which the rats received saline, 5 or 25 mg/kg of antimiR-208a, or 25 mg/kg of antimiR control every 2 weeks. Body weight analysis indicated that Dahl rats on the HS diet exhibited significant reductions in weight gain compared with the controls on the LS diet, whereas the HS/antimiR-208a-treated rats maintained their increase in weight gain (FIG. 1C). Functional assessment using echocardiography indicated that antimiR-208a-treated rats exhibited a significant dose-dependent reduction in isovolumic relaxation time (IVRT) compared to HS/saline controls, as well as a normalization of the mitral valve early to active filling velocity ratio (MV E/A) compared to HS/saline controls eight weeks after the HS diet (FIG. 1D). Time-dependent echocardiography performed every 2 weeks after the HS diet showed significant reductions in isovolumic relaxation time in anti-miR-208a-treated rats compared with HS/saline controls (data not shown). Quantification of cardiomyocyte size showed a significant reduction in cardiomyocyte hypertrophy following treatment with antimiR-208a (FIG. 1E-F). Additionally, antimiR-208a treatment reduced periarteriolar fibrosis induced by HS diet as assessed by quantification of Picrosirius Red staining (FIG. 1E-F),

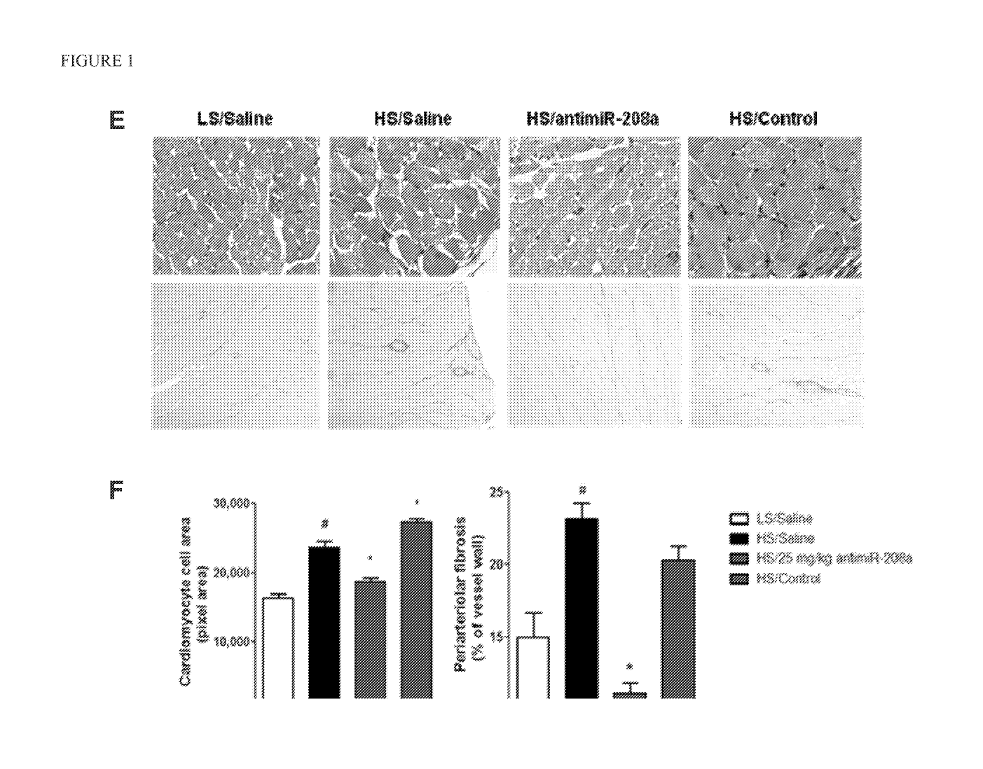

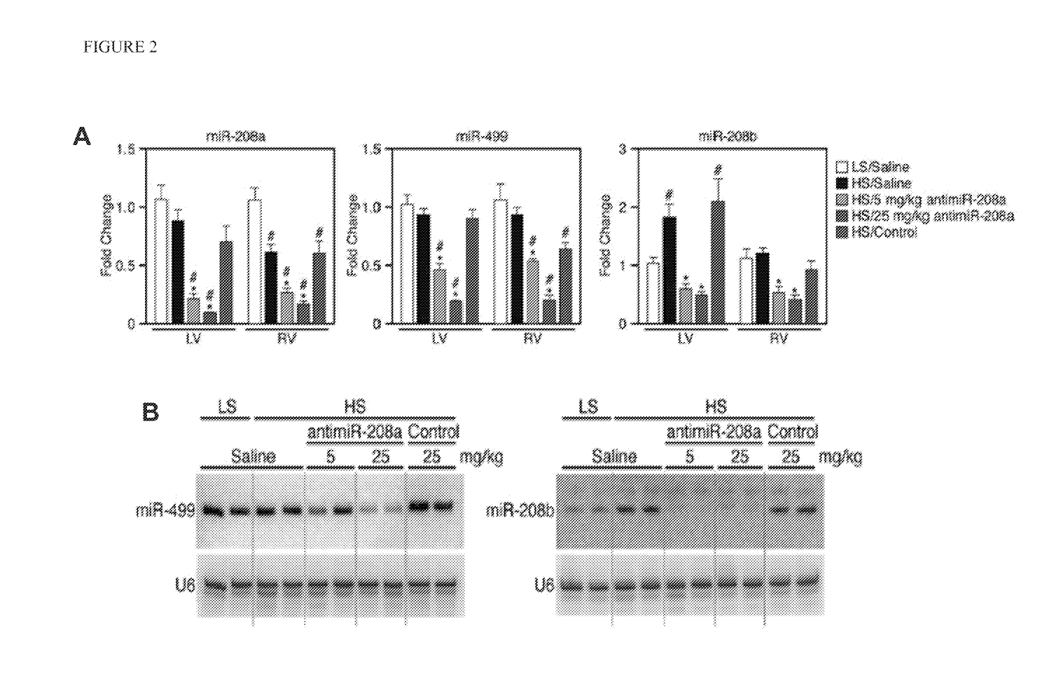

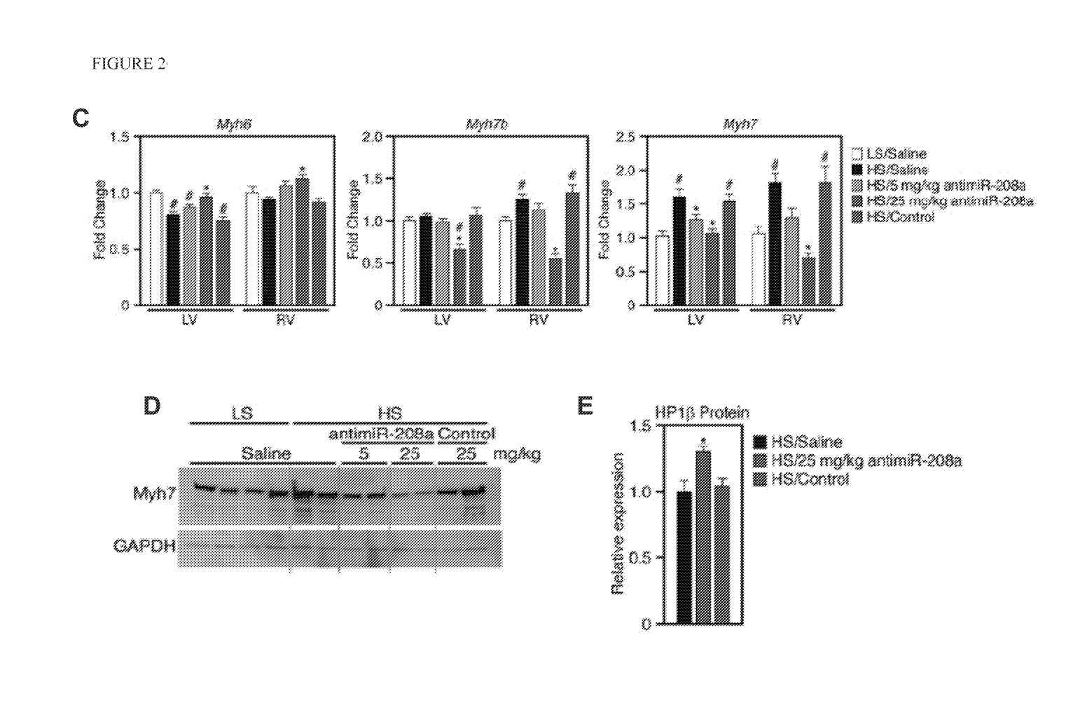

Anti-miR-208a caused a dose-dependent inhibition of miR-208a in both left and right ventricles 2 weeks after the last injection with anti-miR-208a, whereas a control oligo showed no difference compared with saline (FIG. 2A). miR-499 also showed a dose-dependent decrease in expression after sustained inhibition of miR-208a (FIG. 2A). miR-208b was induced in both HS/saline- and HS/control-treated animals, paralleling the upregulation of β-myosin heavy chain; however, anti-miR-208a treatment resulted in a dose-dependent decrease in miR-208b levels (FIG. 2A). This regulation of miR-499 and miR-208b was confirmed by Northern blot analysis (FIG. 2B).

To assess the regulation of the myosin host genes, we examined Myh6, Myh7, and Myh7b mRNA levels. Myh7 was significantly increased in response to HS in both the HS/saline and HS/control groups. This increase was dose-dependently blunted in response to anti-miR-208a. Additionally, anti-miR-208a treatment normalized the decreased expression of Myh6 mRNA observed in both the HS/saline and HS/control groups (FIG. 2C). Expression of Myh7b mirrored miR-499 levels, exhibiting a dose-dependent reduction on anti-miR-208a treatment. Furthermore, the dose-dependent regulation of Myh7 was confirmed by Western blot (FIG. 2D). In addition, anti-miR-208a treatment resulted in a depression of the previously characterized miR-208a target, HP1β, further suggesting that anti-miR-208a action occurs through miR-208a inhibition (FIG. 2E).

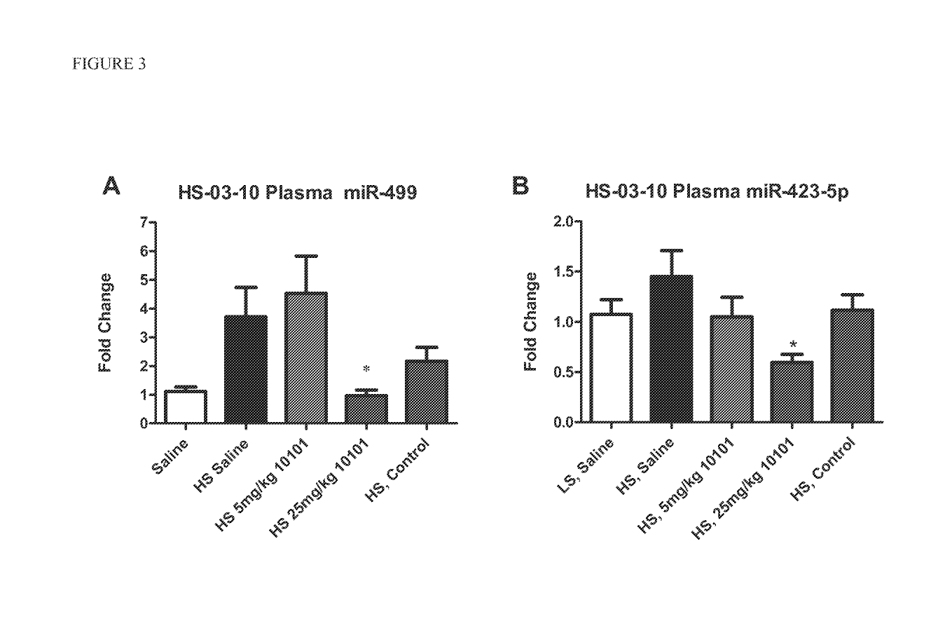

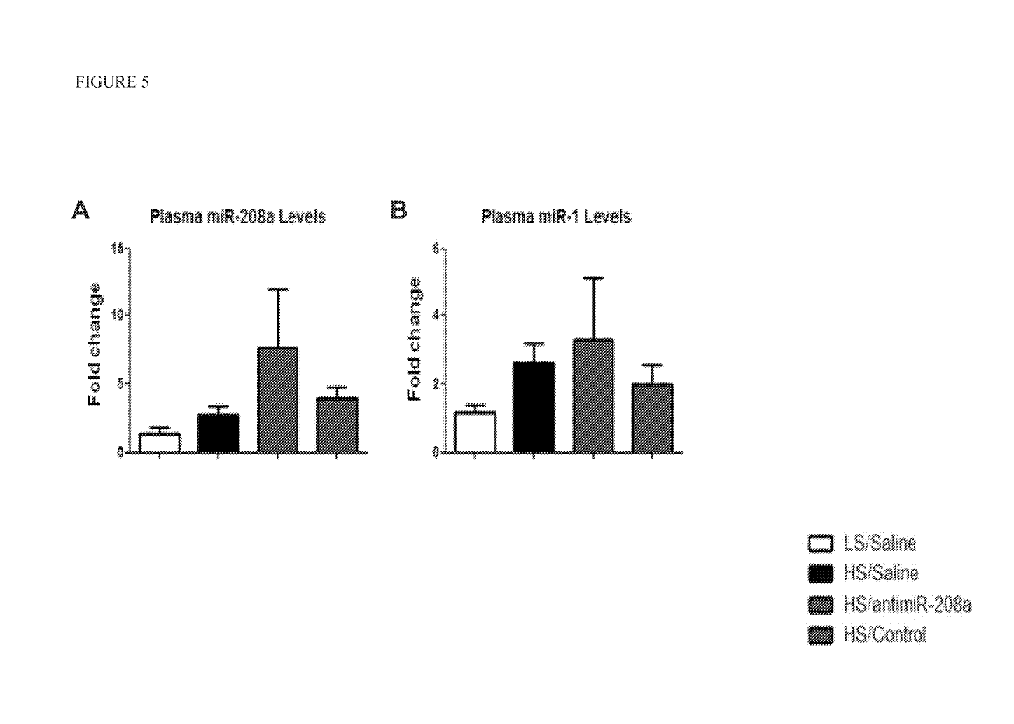

Detection of miRNAs in plasma during various disease settings is showing increasing diagnostic promise (Cortez M A and Calin G A (2009) Expert Opin Biol Ther. 9, 703-711). To determine whether there is a specific miRNA that correlates with antimiR-208a efficacy, we examined a panel of muscle related miRNAs during HS treatment. Strikingly, miR-499, although showing only modest increases in plasma detection under high salt, was significantly reduced in antimiR-208a-treated animals, suggesting miR-499 can act as a plasma-based marker for antimiR-208a efficacy (FIG. 3). Additionally, miR-423-5p plasma levels, which were previously correlated to human heart failure (Tijsen, A. J., et al. (2010) Circ Res 106, 1035-1039), were found to be reduced in animals treated with antimiR-208a (FIG. 3). Other muscle-specific miRNAs did not show significant differences between the groups tested (FIG. 5).

The results of this study indicate that therapeutic inhibition of miR-208 leads to a reduction in cardiac remodeling, which coincides with a significant improvement in survival and cardiac function during heart disease. The efficacy of miR-208a inhibition in the heart can be monitored by measuring plasma levels of miR-499 and miR-423-5p as the plasma levels of these miRNAs correlated with miR-208a knockdown in heart tissue by an antimiR-208 oligonucleotide. Specifically plasma miRNA analysis showed that anti-miR-208a treatment results in a diminution of miR-499 and miR-423-5p in blood serum, which parallels the decrease in cardiac expression of Myh7b/miR-499 in response to anti-miR-208a treatment. Given the correlation between plasma-based miR-499/miR-423-5p levels and the efficacy of anti-miR-208a, these data suggest that plasma miR-499 and miR-423-5p levels would serve as biomarkers of effective delivery of anti-miR-208a to the heart when moving into patients.

### Specific Methods

Animals and Delivery of LNA-Modified antimiRs.

The LNA-antimiR oligonucleotides were synthesized at miRagen Therapeutics, Inc. as unconjugated and fully phosphorothiolated oligonucleotides perfectly complementary to the 5′ region of the mature miR-208a sequence. The LNA control oligonucleotide consisted of a sequence directed against a C. elegans specific miRNA. Unless otherwise indicated, in vivo delivery of the oligonucleotide chemistries was achieved by low pressure intravenous (i.v.) injections via the tail vein or subcutaneous injections of either adult male C56B16 mice or adult male Dahl Salt-sensitive rats (Harlan, Indianapolis). All chemistries were dissolved and injected in a comparable end volume of saline after which the animals were examined for obvious side effects of the chemistries. Tissue samples were collected at the indicated timepoints for molecular or histological examination. Dahl rats were maintained on 0.25 NaCl or placed on 4% or 8% NaCl diet at 8 weeks of age (Harlan, Indianapolis).

Quantitative Real-Time PCR Analysis.

For in vivo real-time PCR analysis, RNA was extracted from cardiac tissue using Trizol (Invitrogen) after which two μg RNA from each tissue sample was used to generate cDNA using Super Script II reverse transcriptase per manufacturer's specifications (Invitrogen). To detect the level of miR-208 RT-PCR was performed using the Taqman MicroRNA assay (Applied Biosystems, ABI) according the manufacturer's recommendations, using 10-100 ng of total RNA. The expression of a subset of genes was analyzed by quantitative real time PCR using Taqman probes purchased from ABI.

Northern Blot Analysis.

Total RNA was isolated from cardiac tissue samples by using Trizol reagent (Gibco/BRL). Northern blots to detect microRNAs were performed as described previously described. A U6 probe served as a loading control (IDT). 10 μg of total RNA from cardiomyocytes or heart tissue was loaded on 20% acrylamide denaturing gels and transferred to Zeta-probe GT genomic blotting membranes (Bio-Rad) by electrophoresis. After transfer, the blots were cross-linked and baked at 80° C. for 1 hr. To maximize the sensitivity of miRNA detection, oligonucleotide probes were labeled with the Starfire Oligos Kit (IDT, Coralville, Iowa) and α-³²P dATP (Amersham or Perkin Elmer). Probes were hybridized to the membranes overnight at 39° C. in Rapid-hyb buffer (Amersham), after which they were washed twice for 10 minutes at 39° C. with 0.5×SSC containing 0.1% SDS. The blots were exposed and quantified by PhosphorImager analysis (GE HealthCare Life Sciences) and a U6 probe served as a loading control (ABI). The intensity of the radioactive signal was used to quantify the fold change in expression using a phosphorimager and ImageQuant (Bio-Rad).

Western blot analysis. For Western blot analysis, Myosin was extracted from cardiac cells or tissue as described (H-amalainen, N. & Pette, D. Patterns of myosin isoforms in mammalian skeletal muscle fibres. Microsc Res Tech 30, 381-389 (1995)). MHC isoforms were detected by loading 0.1 ug protein lysate on a 4-15% gradient gel and separated by SDS PAGE and Western blotting was performed with mouse monoclonal anti-myosin (slow, skeletal M8421) (Sigma, Mo.), which is highly specific for Myh7.

Quantitative Real-Time PCR Analysis from Plasma.

RNA from plasma samples was isolated using Trizol LS Reagent (Invitrogen), using the manufacturer's protocol. Prior to RNA isolation, 250 μmol of two different synthetic C. elegans miRNA sequences were added to serve as internal controls for normalization of target miRNAs. The C. elegans sequences used were cel-miR-2 (UAUCACAGCCAGCCAGCUUUGAUGUGC, SEQ ID NO:5), and cel-lin-4 (UCCCUGAGACCUCAAGUGUGA; SEQ ID NO:6) (Dharmacon). The final RNA pellet was re-suspended in a final volume equal to the initial plasma volume and 5 μl was used for subsequent RT-PCR reactions, as described above.

Statistical Analysis.

One-way ANOVA and Newman-Keuls Multiple Comparison Post-test were used to determine significance. P<0.05 was considered statistically significant.

### Example 2

### Plasma miRNAs are Biomarkers of Disease Progression of Heart Failure and Therapeutic Efficacy of antimiR-208 Oligonucleotides

To evaluate plasma biomarkers over time, surgically implanted catheters were used for non-invasive bi-weekly blood draws in the Dahl salt-sensitive rat model of congestive heart failure described in Example 1. Changes in plasma miRNA biomarkers with disease progression and therapeutic treatment with an LNA-modified antisense oligonucleotide targeting miR-208a (M−10101; SEQ ID NO: 12) were evaluated. The flowing experimental groups were tested:

| Group | Strain | Diet | Treatment |
|---|---|---|---|
| Group 1 | Dahl SS | Regular Chow | Saline subcutaneously |
| Group 2 | Dahl SS | 4% NaCl Diet | Saline subcutaneously |
| Group 3 | Dahl SS | 4% NaCl Diet | M-10101 antimiR (25 mg/kg) |
| | | | subcutaneously |
| Group 4 | Dahl SS | 4% NaCl Diet | M-10591 control (25 mg/kg) |
| | | | subcutaneously |

The M-10591 oligonucleotide (SEQ ID NO:13) targets miR-67 from C. elegans, which is not expressed in mammalian tissue, and therefore was used as a control. Dahl salt-sensitive rats were implanted with catheters at week zero and baseline blood was taken. Following catheter implant surgery, high-salt diet was initiated. One week following the initiation of the 4.0% NaCl diet, rats were dosed subcutaneously (s.c.) with saline or antimiRs at 25 mg/kg once every 2 weeks. On alternating weeks (non-injection weeks) 1.5 mls of blood was drawn from the catheter for plasma isolation. Animals were sacrificed 8 weeks after the initial injection.

Plasma and tissue molecular endpoints were evaluated using Taqman RT-PCR assays. Specifically, RNA from plasma samples was isolated using Trizol LS (Invitrogen). Synthetic C. elegans miRNA sequences for lin-4 and miR-2 were added at a constant concentration to each sample to monitor purification efficiency and for normalization. Tissue RNA was isolated using standard Trizol RNA isolation. Standard ABI Taqman miRNA assay protocols were used to determine relative miRNA levels.

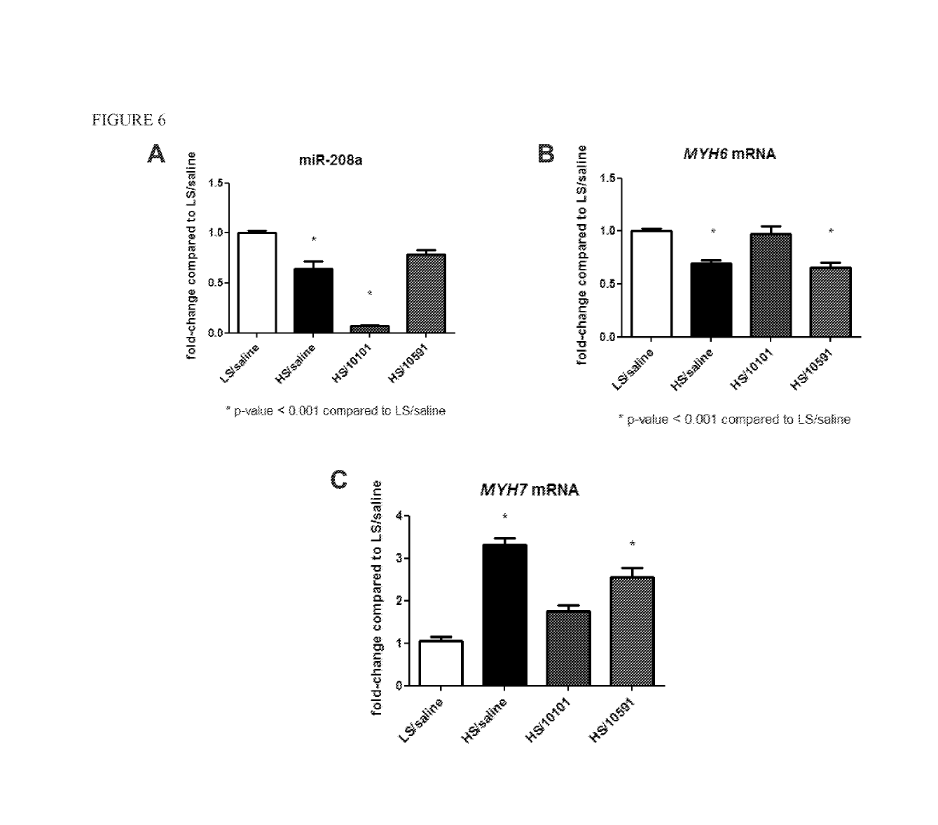

To evaluate the efficacy of antimiR-208 treatment, cardiac tissue markers were measured. High-salt diet (HS) resulted in a significant decrease in miR-208a levels. Treatment with the M-10101 antimiR-208a oligonucleotide resulted in greater inhibition of miR-208a in cardiac tissue compared to HS/Saline group (FIG. 6A). Examination of Myh6 expression, the host gene for miR-208a, revealed a significant decrease in animals on the HS diet receiving either saline or the M-10591 control oligonucleotide as compared to animals in the low-salt (LS)/saline group (FIG. 6B). In contrast, treatment with the antimiR-208a prevented the high-salt diet-induced decrease in Myh6 expression (FIG. 6B). Expression of the Myh7gene was significantly increased in animals in the HS/saline and HS/control oligonucleotide groups as compared to LS/Saline group (FIG. 6C). AntimiR-208a treatment blunted the hypertension-induced increase in Myh7 expression (FIG. 6C). These results show that the antimiR-208 oligonucleotide treatment was effective at the molecular level in reducing gene expression changes induced by hypertensive conditions.

Changes in levels of various miRNAs in plasma during progression of hypertension in Dahl salt-sensitive rats (low-salt diet/saline vs. high-salt/saline groups) was measured by miRNA Taqman array card analysis and confirmed by real-time PCR analysis after 8 weeks on either the low or high salt diet (Table 1). These plasma miRNAs can serve as biomarkers to assess disease severity and heart failure progression over time.

TABLE 1
Fold change of plasma miRNAs in Healthy Rats vs. rats with hypertension-
induced heart failure (LS-Saline vs. HS-Saline).

| Up-regulated | | Down Regulated | |
|---|---|---|---|
| | Fold | | Fold |
| miRNA | Change | miRNA | Change |
| hsa-miR-16 (SEQ ID NO: 26) | 4.02 | hsa-miR-204 (SEQ ID NO: 68) | 3.02 |
| hsa-miR-223 (SEQ ID NO: 4) | 2.61 | hsa-miR-199b-5p (SEQ ID NO: 112) | 2.93 |
| hsa-miR-320 (SEQ ID NO: 30) | 2.55 | hsa-miR-125a-5p (SEQ ID NO: 67) | 2.24 |
| hsa-miR-150 (SEQ ID NO: 114) | 2.53 | hsa-miR-143 (SEQ ID NO: 64) | 1.92 |
| hsa-miR-378 (SEQ ID NO: 115) | 2.23 | hsa-miR-195 (SEQ ID NO: 34) | 1.48 |
| hsa-miR-92a (SEQ ID NO: 50) | 2.11 | | |
| hsa-miR-423-5p (SEQ ID NO: 2) | 2.10 | | |
| hsa-miR-133a (SEQ ID NO: 3) | 2.06 | | |
| hsa-miR-22 (SEQ ID NO: 116) | 1.78 | | |
| hsa-miR-21 (SEQ ID NO: 117) | 1.68 | | |
| hsa-miR-210 (SEQ ID NO: 113) | 1.68 | | |
| hsa-miR-20b (SEQ ID NO: 17) | 1.64 | | |
| hsa-miR-499 (SEQ ID NO: 1) | 1.62 | | |
| hsa-miR-378* (SEQ ID NO: 118) | 1.60 | | |
| hsa-miR-106b (SEQ ID NO: 25) | 1.58 | | |
| hsa-miR-155 (SEQ ID NO: 90) | 1.47 | | |
| hsa-miR-93 (SEQ ID NO: 20) | 1.34 | | |

Changes in plasma miRNA levels between rats suffering from hypertension-induced heart failure (H S/saline group) and rats treated with an antimiR-208a oligonucleotide (HS/M-10101) were assessed at 8 weeks by both miRNA Taqman array card analysis (Tables 2 and 3) and real-time PCR analysis (Table 4).

TABLE 2
Fold change of plasma miRNAs that are decreased in
response to therapeutic treatment in rats with
hypertension-induced heart failure
(HS-saline/HS-10101)
measured by miRNA Taqman array card analysis.

| | | SEQ | |
|---|---|---|---|
| | Mature miRNA Sequence | ID | Fold- |
| miRNA | (5′ to 3′) | NO | Change |
| hsa-miR-542-3p | UGUGACAGAUUGAUAACUGAAA | 14 | 11.10 |
| hsa-miR-185 | UGGAGAGAAAGGCAGUUCCUGA | 15 | 7.02 |
| hsa-miR-199a-5p | CCCAGUGUUCAGACUACCUGUUC | 16 | 4.95 |
| hsa-miR-20b | CAAAGUGCUCAUAGUGCAGGUAG | 17 | 4.87 |
| hsa-miR-423-5p | UGAGGGGCAGAGAGCGAGACUUU | 2 | 4.18 |
| hsa-miR-451 | AAACCGUUACCAUUACUGAGUU | 18 | 3.28 |
| hsa-miR-140-5p | CAGUGGUUUUACCCUAUGGUAG | 19 | 3.23 |
| hsa-miR-93 | CAAAGUGCUGUUCGUGCAGGUAG | 20 | 2.93 |
| hsa-miR-27a | UUCACAGUGGCUAAGUUCCGC | 21 | 2.74 |
| hsa-miR-365 | UAAUGCCCCUAAAAAUCCUUAU | 22 | 2.61 |
| hsa-miR-148a | UCAGUGCACUACAGAACUUUGU | 23 | 2.49 |
| hsa-miR-20a | UAAAGUGCUUAUAGUGCAGGUAG | 24 | 2.43 |
| hsa-miR-133a | UUUGGUCCCCUUCAACCAGCUG | 3 | 2.21 |
| hsa-miR-106b | UAAAGUGCUGACAGUGCAGAU | 25 | 2.15 |
| hsa-miR-16 | UAGCAGCACGUAAAUAUUGGCG | 26 | 2.04 |
| hsa-miR-18a | UAAGGUGCAUCUAGUGCAGAUAG | 27 | 2.01 |
| hsa-miR-26b | UUCAAGUAAUUCAGGAUAGGU | 28 | 1.98 |
| hsa-miR-192 | CUGACCUAUGAAUUGACAGCC | 29 | 1.92 |
| hsa-miR-320a | AAAAGCUGGGUUGAGAGGGCGA | 30 | 1.89 |
| hsa-miR-223 | UGUCAGUUUGUCAAAUACCCCA | 4 | 1.88 |
| hsa-miR-744 | UGCGGGGCUAGGGCUAACAGCA | 31 | 1.83 |
| hsa-miR-301a | CAGUGCAAUAGUAUUGUCAAAGC | 32 | 1.79 |
| hsa-miR-188-5p | CAUCCCUUGCAUGGUGGAGGG | 33 | 1.76 |
| hsa-miR-195 | UAGCAGCACAGAAAUAUUGGC | 34 | 1.73 |
| hsa-miR-122 | UGGAGUGUGACAAUGGUGUUUG | 35 | 1.59 |
| hsa-miR-197 | UUCACCACCUUCUCCACCCAGC | 36 | 1.52 |
| hsa-miR-125b-5p | UCCCUGAGACCCUAACUUGUGA | 37 | 1.51 |
| hsa-miR-486-5p | UCCUGUACUGAGCUGCCCCGAG | 38 | 1.49 |
| hsa-miR-484 | UCAGGCUCAGUCCCCUCCCGAU | 39 | 1.46 |
| hsa-miR-184 | UGGACGGAGAACUGAUAAGGGU | 40 | 1.42 |
| hsa-miR-17 | CAAAGUGCUUAGAGUGCAGGUAG | 41 | 1.41 |
| hsa-miR-324-5p | CGCAUCCCCUAGGGCAUUGGUGU | 42 | 1.39 |
| hsa-miR-182 | UUUGGCAAUGGUAGAACUCACACU | 43 | 1.39 |
| hsa-miR-181c | AACAUUCAACCUGUCGGUGAGU | 44 | 1.37 |
| hsa-miR-29c | UAGCACCAUUUGAAAUCGGUUA | 45 | 1.34 |
| hsa-miR-25 | CAUUGCACUUGUCUCGGUCUGA | 46 | 1.33 |
| hsa-miR-218 | UUGUGCUUGAUCUAACCAUGU | 47 | 1.32 |
| hsa-miR-30a | UGUAAACAUCCUCGACUGGAAG | 48 | 1.28 |
| hsa-miR-30b | UGUAAACAUCCUACACUCAGCU | 49 | 1.21 |
| hsa-miR-92a | UAUUGCACUUGUCCCGGCCUGU | 50 | 1.20 |
| hsa-miR-340-5p | UUAUAAAGCAAUGAGACUGAUU | 51 | 1.19 |
| hsa-miR-27b | UUCACAGUGGCUAAGUUCUGC | 52 | 1.18 |
| hsa-miR-24 | UGGCUCAGUUCAGCAGGAACAG | 53 | 1.17 |
| hsa-miR-103a | AGCAGCAUUGUACAGGGCUAUGA | 54 | 1.16 |
| hsa-miR-203 | GUGAAAUGUUUAGGACCACUAG | 55 | 1.13 |
| hsa-miR-222 | AGCUACAUCUGGCUACUGGGU | 56 | 1.11 |
| hsa-miR-101a | UACAGUACUGUGAUAACUGAA | 57 | 1.11 |
| hsa-let-7b | UGAGGUAGUAGGUUGUGUGGUU | 58 | 1.09 |
| hsa-miR-335-5p | UCAAGAGCAAUAACGAAAAAUGU | 59 | 1.07 |

TABLE 3
Fold change of plasma miRNAs that are increased
in response to therapeutic treatment in rats with
hypertension-induced heart failure
(HS-10101/HS-Saline)
measured by miRNA Taqman array card analysis.

| | | SEQ | |
|---|---|---|---|
| | Mature miRNA Sequence | ID | Fold- |
| miRNA | (5' to 3') | NO | Change |
| hsa-miR-142-3p | UGUAGUGUUUCCUACUUUAUGGA | 60 | 25.04 |
| hsa-miR-296-5p | AGGGCCCCCCCUCAAUCCUGU | 61 | 18.29 |
| hsa-miR-125a-3p | ACAGGUGAGGUUCUUGGGAGCC | 62 | 12.16 |
| hsa-miR-196b | UAGGUAGUUUCCUGUUGUUGGG | 63 | 11.73 |
| hsa-miR-143 | UGAGAUGAAGCACUGUAGCUC | 64 | 8.47 |
| hsa-miR-28 | AAGGAGCUCACAGUCUAUUGAG | 65 | 6.61 |
| hsa-miR-107 | AGCAGCAUUGUACAGGGCUAUCA | 66 | 5.54 |
| hsa-miR-125a-5p | UCCCUGAGACCCUUUAACCUGUGA | 67 | 5.34 |
| hsa-miR-204 | UUCCCUUUGUCAUCCUAUGCCU | 68 | 4.61 |
| hsa-miR-19b | UGUGCAAAUCCAUGCAAAACUGA | 69 | 3.27 |
| hsa-miR-199a-3p | ACAGUAGUCUGCACAUUGGUUA | 70 | 3.14 |
| hsa-let-7g | UGAGGUAGUAGUUUGUACAGUU | 71 | 3.02 |
| hsa-miR-181a | AACAUUCAACGCUGUCGGUGAGU | 72 | 3.00 |
| hsa-miR-449a | UGGCAGUGUAUUGUUAGCUGGU | 73 | 2.99 |
| hsa-miR-29b | UAGCACCAUUUGAAAUCAGUGUU | 74 | 2.67 |
| hsa-miR-487b | AAUCGUACAGGGUCAUCCACUU | 75 | 2.67 |
| hsa-miR-99b | CACCCGUAGAACCGACCUUGCG | 76 | 2.63 |
| hsa-miR-130a | CAGUGCAAUGUUAAAAGGGCAU | 77 | 2.30 |
| hsa-miR-574-3p | CACGCUCAUGCACACACCCACA | 78 | 2.20 |
| hsa-miR-339-5p | UCCCUGUCCUCCAGGAGCUCACG | 79 | 2.18 |
| hsa-miR-302a | UAAGUGCUUCCAUGUUUUGGUGA | 80 | 2.10 |
| hsa-miR-145 | GUCCAGUUUUCCCAGGAAUCCCU | 81 | 2.07 |
| hsa-miR-152 | UCAGUGCAUGACAGAACUUGG | 82 | 2.05 |
| hsa-miR-126-5p | CAUUAUUACUUUUGGUACGCG | 83 | 2.01 |
| (miR-126*) | | | |
| hsa-miR-142-5p | CAUAAAGUAGAAAGCACUACU | 84 | 1.98 |
| hsa-let-7d | AGAGGUAGUAGGUUGCAUAGUU | 85 | 1.98 |
| hsa-miR-19a | UGUGCAAAUCUAUGCAAAACUGA | 86 | 1.90 |
| hsa-miR-340-3p | UCCGUCUCAGUUACUUUAUAGC | 87 | 1.86 |
| hsa-miR-186 | CAAAGAAUUCUCCUUUUGGGCU | 88 | 1.83 |
| hsa-miR-128a | UCACAGUGAACCGGUCUCUUU | 89 | 1.75 |
| hsa-miR-155 | UUAAUGCUAAUCGUGAUAGGGGU | 90 | 1.73 |
| hsa-miR-532-3p | CCUCCCACACCCAAGGCUUGCA | 91 | 1.72 |
| hsa-let-7e | UGAGGUAGGAGGUUGUAUAGUU | 92 | 1.66 |
| hsa-miR-187 | UCGUGUCUUGUGUUGCAGCCGG | 93 | 1.60 |
| hsa-miR-214 | ACAGCAGGCACAGACAGGCAGU | 94 | 1.57 |
| hsa-let-7c | UGAGGUAGUAGGUUGUAUGGUU | 95 | 1.54 |
| hsa-miR-331-3p | GCCCCUGGGCCUAUCCUAGAA | 96 | 1.53 |
| hsa-miR-31 | AGGCAAGAUGCUGGCAUAGCU | 97 | 1.45 |
| hsa-miR-26a | UUCAAGUAAUCCAGGAUAGGCU | 98 | 1.39 |
| hsa-miR-30c | UGUAAACAUCCUACACUCUCAGC | 99 | 1.37 |
| hsa-miR-375 | UUUGUUCGUUCGGCUCGCGUGA | 100 | 1.32 |
| hsa-miR-29a | UAGCACCAUCUGAAAUCGGUUA | 101 | 1.31 |
| hsa-miR-30d | UGUAAACAUCCCCGACUGGAAG | 102 | 1.29 |
| hsa-miR-126 | UCGUACCGUGAGUAAUAAUGCG | 103 | 1.27 |
| hsa-miR-130b | CAGUGCAAUGAUGAAAGGGCAU | 104 | 1.26 |
| hsa-miR-191 | CAACGGAAUCCCAAAAGCAGCUG | 105 | 1.23 |
| hsa-miR-146a | UGAGAACUGAAUUCCAUGGGUU | 106 | 1.17 |
| hsa-miR-342-3p | UCUCACACAGAAAUCGCACCCGU | 107 | 1.15 |
| hsa-miR-139-5p | UCUACAGUGCACGUGUCUCCAG | 108 | 1.14 |
| hsa-miR-138 | AGCUGGUGUUGUGAAUCAGGCCG | 109 | 1.08 |
| hsa-miR-324-3p | ACUGCCCCAGGUGCUGCUGG | 110 | 1.07 |
| hsa-miR-30e | UGUAAACAUCCUUGACUGGAAG | 111 | 1.06 |

TABLE 4
Fold change of plasma miRNAs in rats with hypertension-induced heart failure vs.
treated rats (HS-Saline vs. HS-10101) as confirmed by Real Time PCR analysis.

| Up-regulated | | Down Regulated | |
|---|---|---|---|
| | Fold | | Fold |
| miRNA | Change | miRNA | Change |
| hsa-miR-19b (SEQ ID NO: 69) | 2.68 | hsa-miR-16 (SEQ ID NO: 26) | 3.45 |
| hsa-miR-199b-5p | 2.39 | hsa-miR-320 (SEQ ID NO: 30) | 3.16 |
| CCCAGUGUUUAGACUAUCUGUUC | | | |
| (SEQ ID NO: 112) | | | |
| hsa-miR-204 (SEQ ID NO: 68) | 2.18 | hsa-miR-223 (SEQ ID NO: 4) | 3.02 |
| hsa-miR-145 (SEQ ID NO: 81) | 2.17 | hsa-miR-93 (SEQ ID NO: 20) | 2.87 |
| hsa-miR-195 (SEQ ID NO: 34) | 2.10 | hsa-miR-106b (SEQ ID NO: 25) | 2.57 |
| hsa-miR-125a-5p (SEQ ID NO: 67) | 2.05 | hsa-miR-423-5p (SEQ ID NO: 2) | 2.52 |
| hsa-miR-143 (SEQ ID NO: 64) | 1.53 | hsa-miR-185 (SEQ ID NO: 15) | 2.35 |
| hsa-miR-214 (SEQ ID NO: 94) | 1.49 | hsa-miR-92a (SEQ ID NO: 50) | 2.32 |
| | | hsa-miR-210 | 2.31 |
| | | CUGUGCGUGUGACAGCGGCUGA | |
| | | (SEQ ID NO: 113) | |
| | | hsa-miR-140 (SEQ ID NO: 19) | 2.13 |
| | | hsa-miR-27a (SEQ ID NO: 21) | 2.10 |
| | | hsa-miR-20b (SEQ ID NO: 17) | 2.10 |
| | | hsa-miR-150 | 2.07 |
| | | UCUCCCAACCCUUGUACCAGUG | |
| | | (SEQ ID NO: 114) | |
| | | hsa-miR-20a (SEQ ID NO: 24) | 1.92 |
| | | hsa-miR-378 | 1.74 |
| | | ACUGGACUUGGAGUCAGAAGG | |
| | | (SEQ ID NO: 115) | |
| | | hsa-miR-22 | 1.50 |
| | | AAGCUGCCAGUUGAAGAACUGU | |
| | | (SEQ ID NO: 116) | |
| | | hsa-miR-21 | 1.48 |
| | | UAGCUUAUCAGACUGAUGUUGA | |
| | | (SEQ ID NO: 117) | |
| | | hsa-miR-378* | 1.38 |
| | | CUCCUGACUCCAGGUCCUGUGU | |
| | | (SEQ ID NO: 118) | |
| | | hsa-miR-122 (SEQ ID NO: 35) | 1.36 |
| | | hsa-miR-126 (SEQ ID NO: 103) | 1.30 |
| | | hsa-miR-133a (SEQ ID NO: 3) | 1.29 |
| | | hsa-miR-499 | 1.26 |
| | | UUAAGACUUGCAGUGAUGUUU | |
| | | (SEQ ID NO: 1) | |

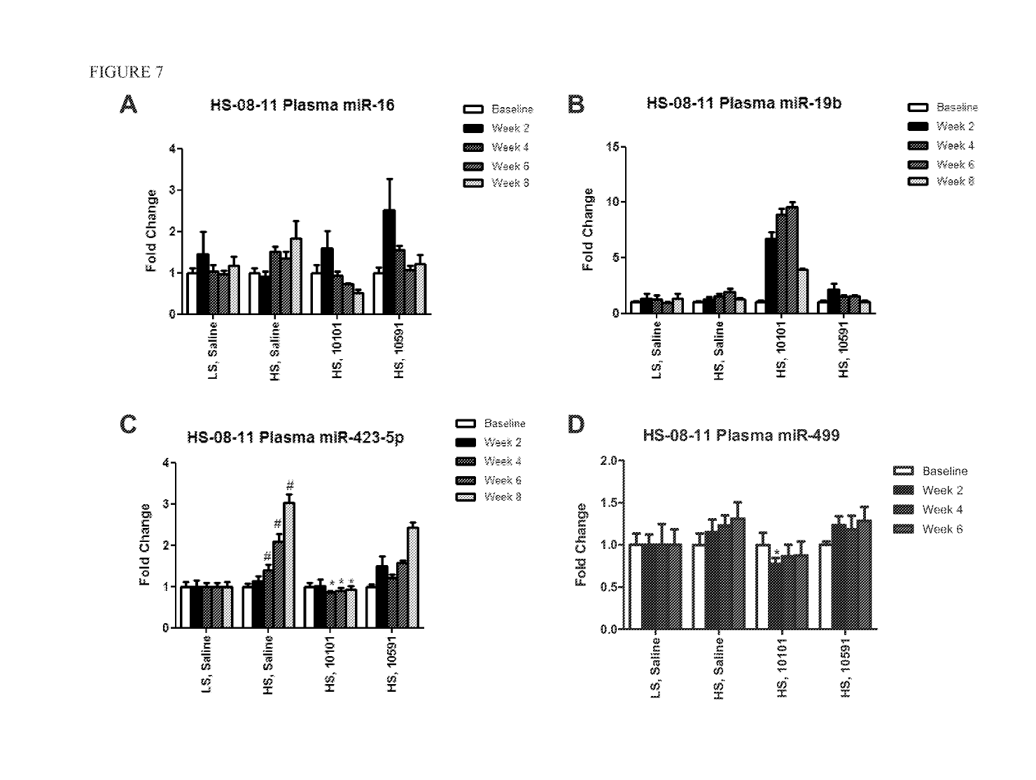

Interestingly, the results of this study show that there are plasma miRNA biomarkers of heart failure progression and severity, therapeutic efficacy of antimiR-208a treatment, and both. All of the disease progression markers were also markers of therapeutic efficacy of antimiR-208 treatment. However, there were several plasma miRNAs, such as miR-19b, miR-199, miR-145, miR-214, miR-185, miR-140, miR-27a, miR-20a, miR-122, and miR-126, that were solely biomarkers of therapeutic efficacy of the antimiR-208a oligonucleotide. Time courses for miR-423-5p, miR-19b, miR-499, and miR-16 plasma levels over the course of the experiment are shown in FIG. 7.

The results from these experiments show that plasma levels (e.g. abundance) of specific miRNAs can serve as surrogate markers of efficacy of cardiac therapeutics, particularly antimiR-208 therapeutics, as well as markers of heart failure progression and severity.

### Example 3

### Both AntimiR and Captopril Treatments Affect Circulating miRs

The Dahl salt-sensitive rat model of congestive heart failure (see Example 1) was used to evaluate the effects of different therapeutic approaches on plasma miRNA levels. Specifically, rats were treated with a LNA-containing antimiR-208 oligonucleotide (M-10101), Captopril (an angiotensin-converting enzyme (ACE) inhibitor), or a combination of the two (M−10101+Captopril).

The following experimental groups were evaluated:

| Group | Strain | N | Diet | Treatment |
|---|---|---|---|---|
| Group 1 | Dahl SS | 6 | Regular Chow | Saline sc/water po |
| Group 2 | Dahl SS | 10 | 6% NaCl Diet | Saline sc/water po |
| Group 3 | Dahl SS | 9 | 6% NaCl Diet | M-10101 (25 mg/kg) sc/water po |
| Group 4 | Dahl SS | 9 | 6% NaCl Diet | Saline sc/Captopril (1.5 mg/kg) po |
| Group 5 | Dahl SS | 9 | 6% NaCl Diet | Saline sc/Captopril (15 mg/kg) po |
| Group 6 | Dahl SS | 9 | 6% NaCl Diet | M-10101 (25 mg/kg) sc/Captopril |
| | | | | (1.5 mg/kg) po |
| Group 7 | Dahl SS | 9 | 6% NaCl Diet | M-10101 (25 mg/kg) sc/Captopril |
| | | | | (15 mg/kg) po |
| Group 8 | Dahl SS | 9 | 6% NaCl Diet | M-10591 (25 mg/kg) sc/Captopril |
| | | | | (15 mg/kg) po |

One week following the initiation of the 6.0% NaCl diet, rats were dosed subcutaneously (sc) with saline or oligonucleotides (M−10101, 5′-CTTTTTGCTCGTCTTA-3′ (SEQ ID NO: 12) or M-10591, 5′-TCCTAGAAAGAGTAGA-3′ (SEQ ID NO:13)) at 25 mg/kg every 2 weeks. Captopril or water were dosed daily by oral gavage. Echocardiography was performed every 2 weeks starting one week post iv dosing. The Captopril dose for rat was selected to match the standard human dose in man. Plasma and tissue molecular endpoints were evaluated using Taqman RT-PCR assays.

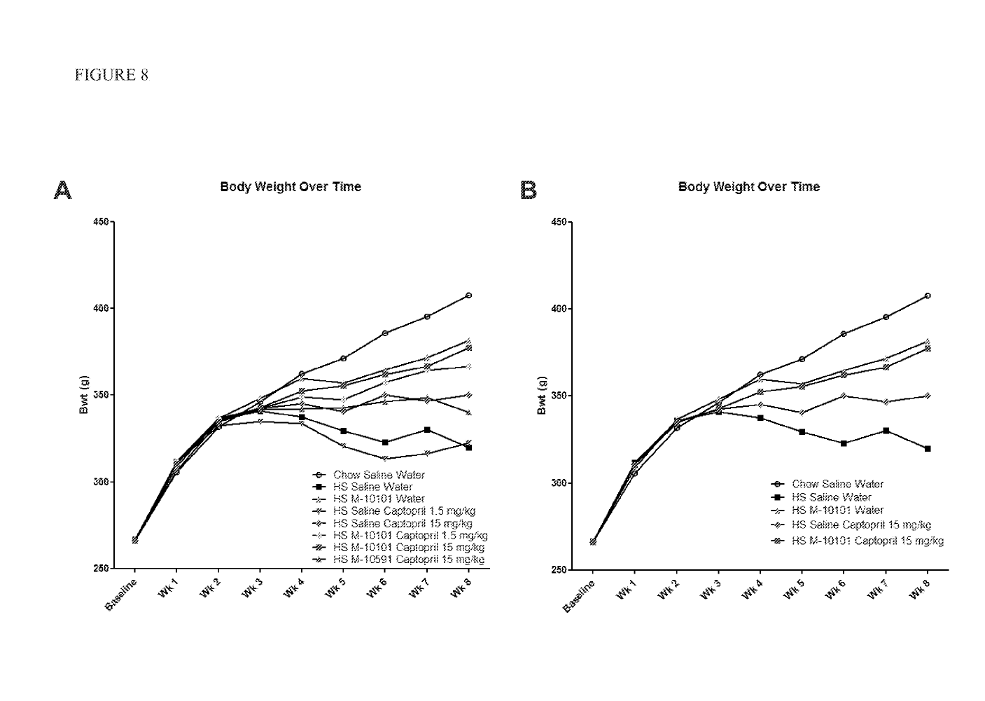

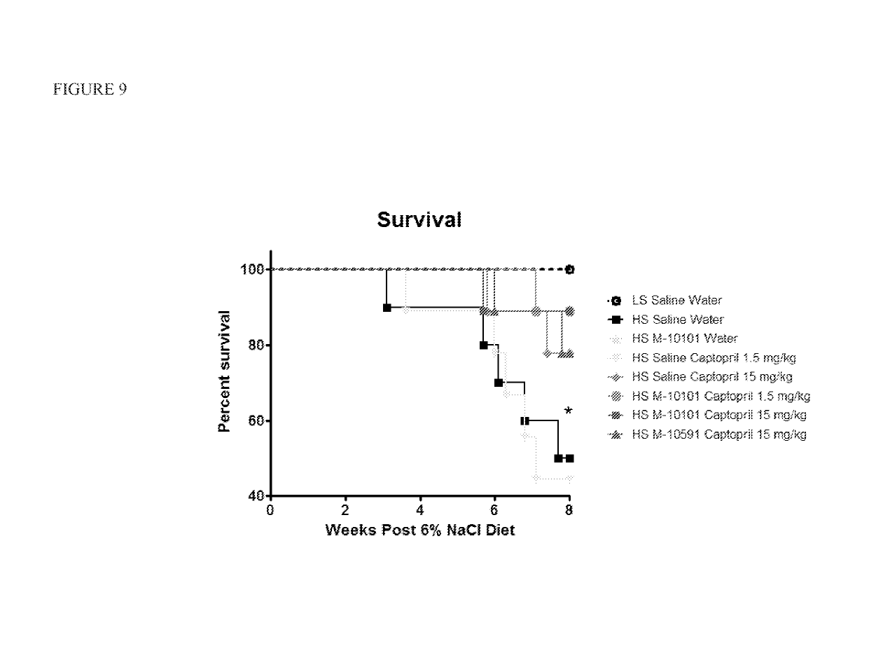

Treatment with the antimiR-208a oligonucleotide (M−10101) partially prevented the body weight loss induced by the high-salt diet (FIG. 8A). In contrast, treatment with the control oligonucleotide (M−10591) had no apparent effect on weight gain. Rats treated with 15 mg/kg Captopril exhibited a lower body weight as compared to animals on a regular, low-salt (LS) diet, but a higher body weight as compared to animals on the high-salt diet (FIG. 8B). Thus, Captopril slightly reduced the high-salt (HS) diet-induced decrease in body weight, but not as effectively as antimiR-208a treatment. Body weight graphs were generated by carrying over final body weights of those rats that died prior to the end of the study. As shown in FIG. 9, only treatment with antimiR-208a (M−10101) alone significantly improved survival following placement of the animals on a high-salt diet (* p<0.05; M-10101, water vs. high-salt diet, saline-treated rats). No mortality was observed in the LS, saline controls or HS, M-10101-treated rats.

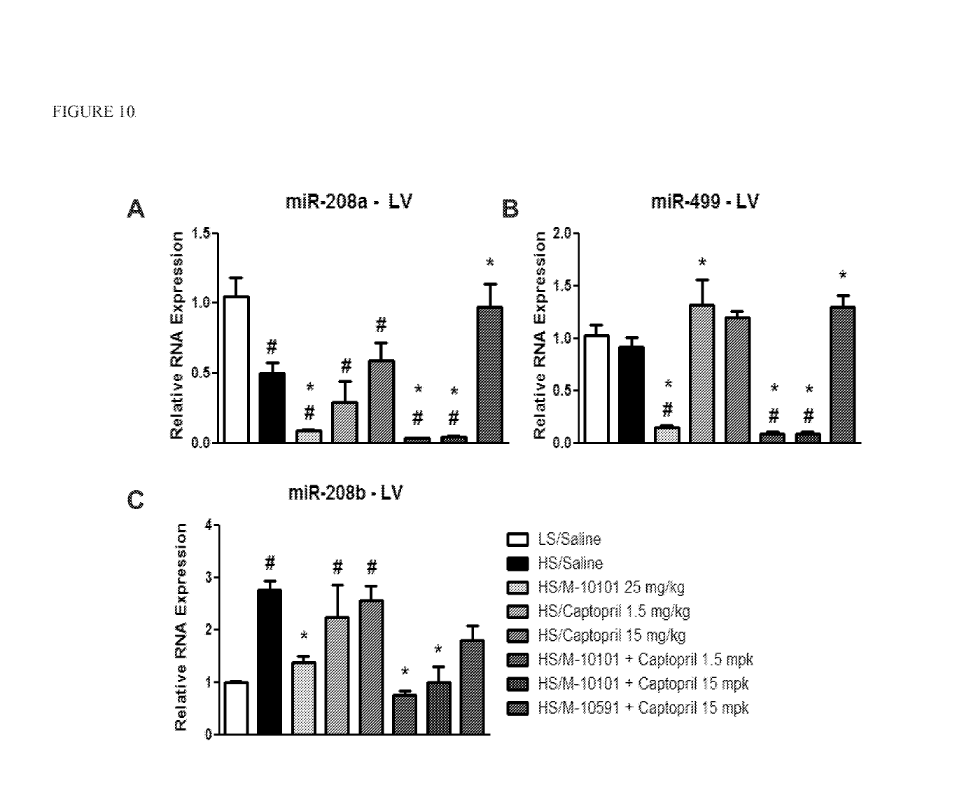

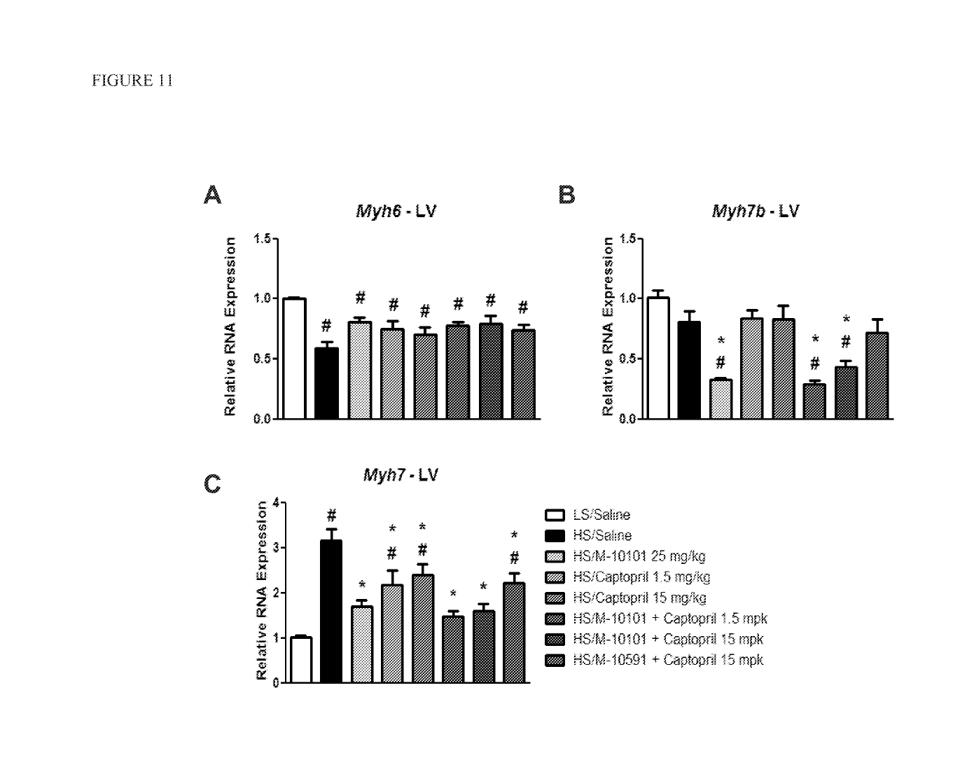

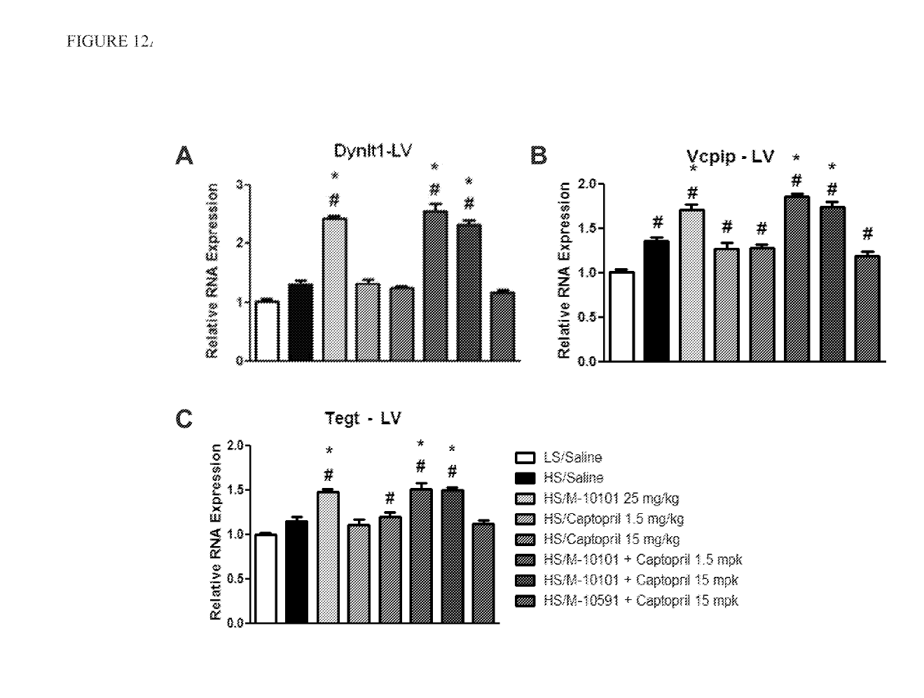

Real time PCR analysis of the myomiRs (miR-208a, miR-499 and miR-208b) showed that the 6% HS diet reduced cardiac levels of miR-208a, and miR-208a levels were further reduced by miR-208a inhibition with M-10101 (FIG. 10A). Treatment with the antimiR-208a oligonucleotide also resulted in a reduction of cardiac levels of miR-499 and miR-208b (FIGS. 10B and C). Real time PCR analysis of the myosin genes showed that the 6% HS diet reduced αMHC (Myh6) in the heart, which was normalized by antimiR-208a oligonucleotide (M−10101) treatment, Captopril treatment, or a combination of both antimiR-208a and Captopril treatment (FIG. 11A). Myh7b levels were specifically reduced in animals receiving the antimiR-208a oligonucleotide (FIG. 11B). However, Captopril treatment alone had no apparent effect on Myh7b expression. Although both Captopril treatment and antimiR-208a oligonucleotide treatment reduced the increase in βMHC (Myh7) expression induced by the HS diet (e.g. cardiac stressor), antimiR-208a oligonucleotide was more effective (FIG. 11C). In fact, βMHC levels in antimiR-208a-treated animals were not significantly different from animals on the LS diet. As shown in FIG. 12, real time PCR analysis of direct target genes of miR-208a, revealed that treatment of animals with the antimiR-208a oligonucleotide, M-10101, produced an increase in expression these genes, indication that M-10101 effectively inhibited miR-208a function.

The results described above demonstrate that both antimiR-208a and the standard of care therapy, Captopril, are effective in reducing both the physiological and molecular changes produced by hypertension. However, antimiR-208a appears to be more effective than Captopril in treating hypertension-induced heart failure in the Dalt salt-sensitive rat model.

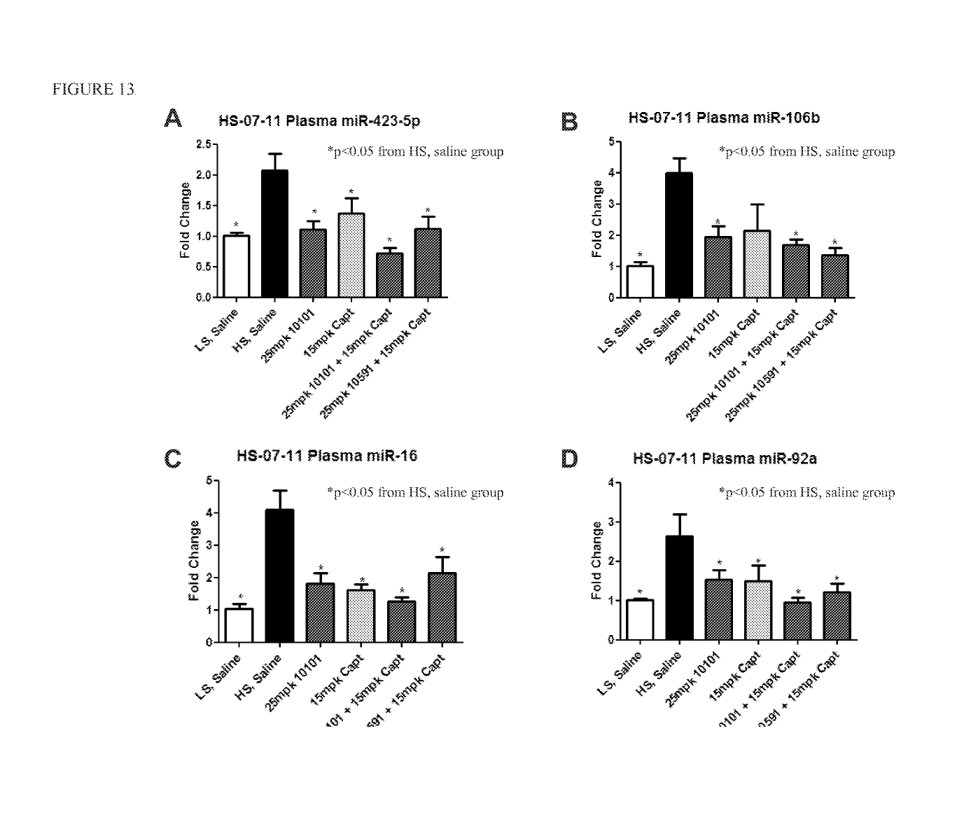

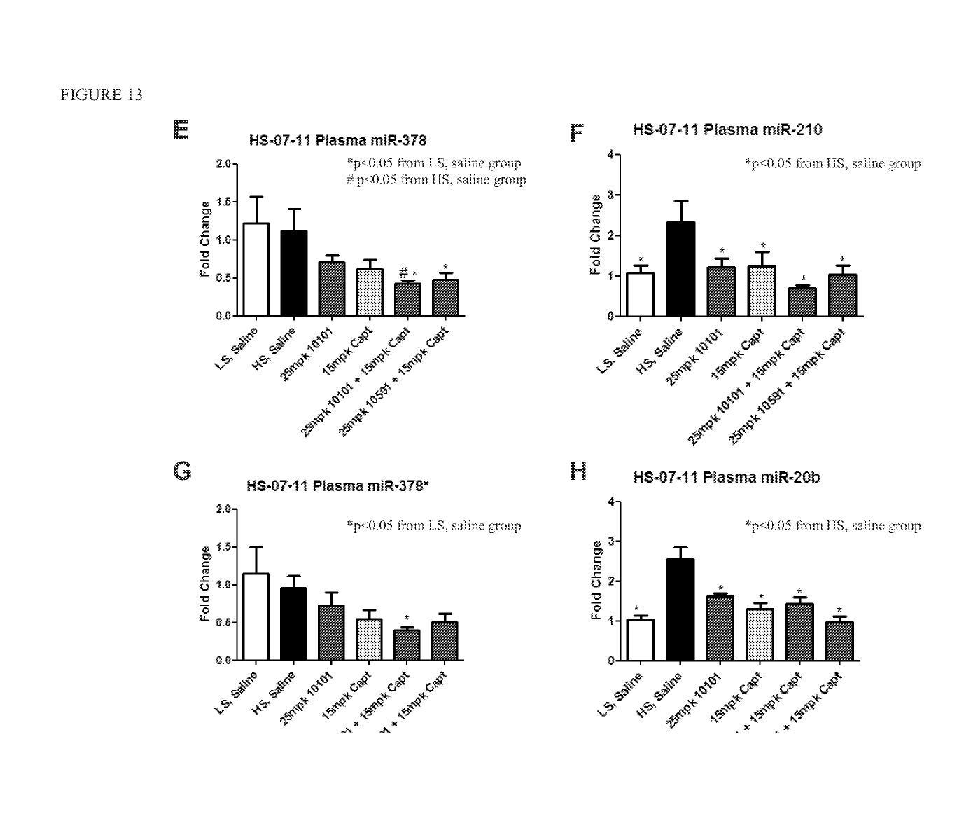

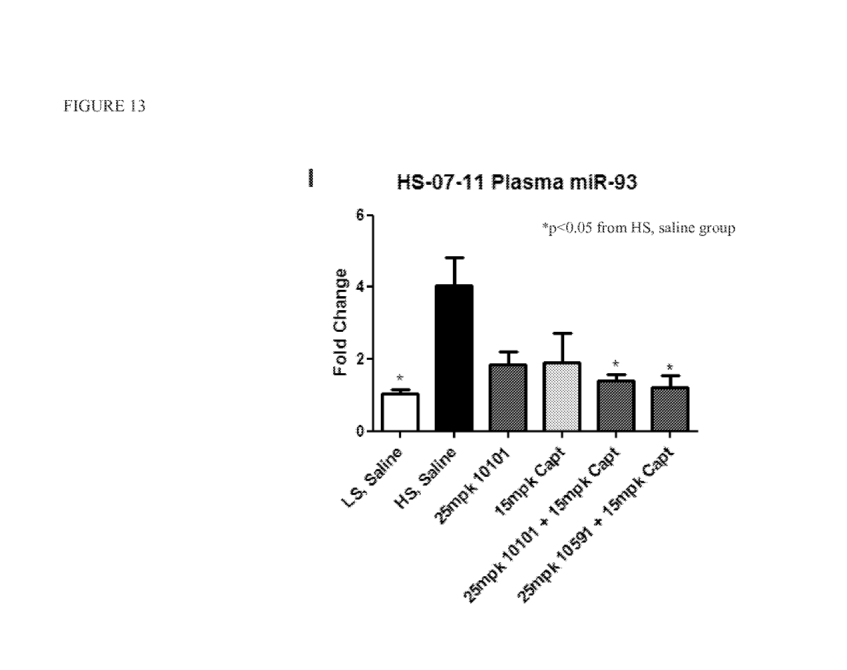

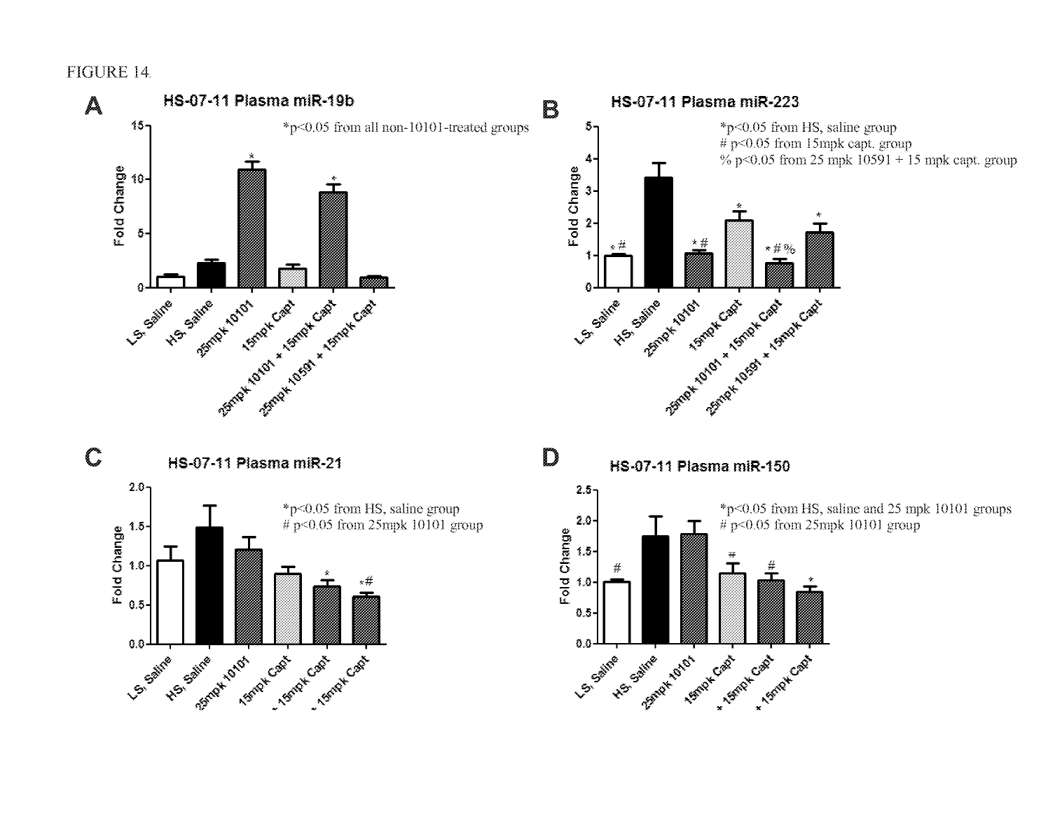

Because plasma levels of various miRNAs correlated with the efficacy of antimiR-208a oligonucleotide treatment (see Example 2), plasma miRNA levels from rats in each of the different treatment groups were measured to determine whether plasma miRNA levels were affected by other cardiac treatments, such as captopril. Plasma levels of several miRNAs appeared to correlate with efficacy of both antimiR-208a oligonucleotide and Captopril therapy (FIG. 13A-I). For example, real time PCR analysis showed that miR-423-5p, miR-106b, miR-16, miR-92a, miR-378, miR-210, miR-378*, miR-20b, and miR-93 were decreased in response to both antimiR-208a and Captopril treatment. Interestingly, plasma levels of miR-378 and miR-378* appeared to be specifically correlated with the combination therapy of both the antimiR and the ACE inhibitor (FIGS. 13E and G). Plasma levels of other miRNAs were correlated with either antimiR-208a oligonucleotide therapy or Captopril therapy. For instance, plasma levels of miR-19B were significantly increased in animals that received the antimiR-208a oligonucleotide (FIG. 14A). Although plasma levels of miR-223 were significantly reduced in response to both types of treatment, a more significant decrease was observed in animals receiving the antimiR-208a oligonucleotide (FIG. 14B). In contrast, levels of miR-21 and miR-150 in plasma were reduced specifically with Captopril treatment (FIGS. 14C-D).

These results show that there is a panel of miRNA for which changes in plasma levels can be observed in response to different cardiac therapies. Interestingly, there appear to be specific plasma miRNAs whose levels correlate with a specific type of cardiac therapy (e.g. miR-208a therapy or ACE inhibitor), whereas levels of other plasma miRNAs appear to correlate with both types of therapies. Still another subset of plasma miRNAs appear to be markers of efficacy of the combined therapy only. Thus, plasma miRNA levels can be used to not only monitor efficacy of cardiac therapies in general, but also to monitor the efficacy of specific therapeutic interventions or combined treatments.

### Specific Methods

Animals and Delivery of LNA-Modified antimiRs.

The LNA-antimiR oligonucleotides were synthesized at miRagen Therapeutics, Inc. as unconjugated and fully phosphorothiolated oligonucleotides perfectly complementary to the 5′ region of the mature miR-208a sequence. The LNA control oligonucleotide consisted of a sequence directed against a C. elegans specific miRNA. Unless else indicated, in vivo delivery of the oligonucleotide chemistries was achieved by low pressure intravenous (i.v.) injections via the tail vein or subcutaneous injection of adult male Dahl Salt-sensitive rats (Harlan, Indianapolis). All chemistries were dissolved and injected in a comparable end volume of saline after which the animals were examined for obvious side effects of the chemistries. Tissue samples were collected at the indicated timepoints for molecular or histological examination. Dahl rats were maintained on 0.25 NaCl or placed on 4%, 6% or 8% NaCl diet at 8 weeks of age (Harlan, Indianapolis).

Quantitative Real-Time PCR Analysis.

For in vivo real-time PCR analysis, RNA was extracted from cardiac tissue using Trizol (Invitrogen) after which two μg RNA from each tissue sample was used to generate cDNA using Super Script II reverse transcriptase per manufacturer's specifications (Invitrogen). To detect the level of miR-208 RT-PCR was performed using the Taqman MicroRNA assay (Applied Biosystems, ABI) according the manufacturer's recommendations, using 10-100 ng of total RNA. The expression of a subset of genes was analyzed by quantitative real time PCR using Taqman probes purchased from AB.

Quantitative real-time PCR analysis from plasma. RNA from plasma samples was isolated using Trizol LS Reagent (Invitrogen), using the manufacturer's protocol. Prior to RNA isolation, 250 μmol of two different synthetic C. elegans miRNA sequences were added to serve as internal controls for normalization of target miRNAs. The C. elegans sequences used were cel-miR-2 (UAUCACAGCCAGCUUUGAUGUGC, SEQ ID NO:5), and cel-lin-4 (UCCCUGAGACCUCAAGUGUGA; SEQ ID NO:6) (Dharmacon). The final RNA pellet was re-suspended in a final volume equal to the initial plasma volume and 5 μl was used for subsequent RT-PCR reactions, as described above.

Statistical Analysis.

One-way ANOVA and Newman-Keuls Multiple Comparison Post-test were used to determine significance. P<0.05 was considered statistically significant.

All publications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

1. A method for evaluating or monitoring the efficacy of a therapeutic intervention for treating a cardiac disorder comprising:
obtaining a plasma or serum sample from a patient receiving the therapeutic intervention;
measuring the level of each miRNA in a panel of miRNAs in the sample, wherein the panel of miRNAs comprises two or more miRNAs listed in any of Tables 2-4; and
comparing the measured levels to pre-determined reference levels or the levels of the miRNAs in a control sample, wherein the comparison is indicative of the therapeutic efficacy of the therapeutic intervention.

2-4. (canceled)

5. The method of claim 1, wherein the panel comprises two or more miRNAs listed in Table 2 or Table 4, and the comparison is a decrease in the measured levels of the miRNAs relative to the pre-determined reference levels or the levels of the miRNAs in the control sample.

6. The method of claim 5, wherein the panel comprises two or more miRNAs selected from hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-126, hsa-miR-133a, and hsa-miR-499.

7. The method of claim 1, wherein the panel comprises two or more miRNAs listed in Table 3 or Table 4, and the comparison is an increase in the measured levels of the miRNAs relative to the pre-determined reference levels or the levels of the miRNAs in the control sample.

8. The method of claim 7, wherein the panel comprises two or more miRNAs selected from hsa-miR-19b, hsa-miR-199b-5p, hsa-miR-204, hsa-miR-145, hsa-miR-195, hsa-miR-125a-5p, hsa-miR-143, and hsa-miR-214.

9. The method of claim 1, wherein the therapeutic intervention is a chemically-modified antisense oligonucleotide targeting a miRNA expressed in heart tissue.

10. (canceled)

11. The method of claim 9, wherein the chemically-modified antisense oligonucleotide targets miR-208a and/or miR-208b.

12. The method of claim 11, wherein the panel comprises two or more miRNAs selected from miR-19b, miR-223, miR-423-5p, miR-106b, miR-16, miR-92a, miR-378, miR-210, miR-378*, miR-20b, and miR-93.

13. The method of claim 12, wherein the panel comprises miR-19b and/or miR-223.

14. (canceled)

15. (canceled)

16. The method of claim 1, wherein the therapeutic intervention is an angiotensin-converting enzyme inhibitor.

17. The method of claim 16, wherein the panel comprises two or more miRNAs selected from miR-150, miR-21, miR-223, miR-423-5p, miR-106b, miR-16, miR-92a, miR-378, miR-210, miR-378*, miR-20b, and miR-93.

18. The method of claim 17, wherein the panel comprises miR-21 and/or miR-150.

19. The method of claim 1, wherein the cardiac disorder is myocardial infarction, pathologic cardiac hypertrophy, heart failure, or hypertension.

20. The method of claim 1, wherein the plasma or serum sample is obtained from the patient at least one week following the start of the therapeutic intervention.

21. (canceled)

22. (canceled)

23. The method of claim 1, further comprising altering the therapeutic intervention based on the comparison.

24. (canceled)

25. A kit comprising miRNA-specific primers for reverse transcribing or amplifying two or more miRNAs selected from any one of Tables 2-4 in a plasma or serum sample from a patient receiving treatment for a cardiac disorder and instructions for measuring said two or more miRNAs for evaluating or monitoring the efficacy of a therapeutic intervention for treating a cardiac disorder in the patient.

26. The kit of claim 25, wherein the kit comprises a miRNA-specific primers for two or more miRNAs selected from hsa-miR-16, hsa-miR-320, hsa-miR-223, hsa-miR-93, hsa-miR-106b, hsa-miR-423-5p, hsa-miR-185, hsa-miR-92a, hsa-miR-210, hsa-miR-140, hsa-miR-27a, hsa-miR-20b, hsa-miR-150, hsa-miR-20a, hsa-miR-378, hsa-miR-22, hsa-miR-21, hsa-miR-378*, hsa-miR-122, hsa-miR-126, hsa-miR-133a, hsa-miR-499, hsa-miR-19b, hsa-miR-199b-5p, hsa-miR-204, hsa-miR-145, hsa-miR-195, hsa-miR-125a-5p, hsa-miR-143, and hsa-miR-214.

27-31. (canceled)

32. The kit of claim 25, further comprising a labeled-nucleic acid probe specific for each miRNA of the kit.

33. (canceled)

34. (canceled)

35. A method of predicting or assessing the level of severity of heart failure or heart failure progression in a patient comprising:
obtaining a plasma or serum sample from a patient having symptoms of heart failure;
measuring the level of each miRNA in a panel of miRNAs in the sample, wherein the panel of miRNAs comprises two or more miRNAs listed in Table 1; and
comparing the measured levels to pre-determined reference levels or the levels of the miRNAs in a control sample, wherein the comparison is indicative of the level of severity of heart failure or heart failure progression in the patient.

36. The method of claim 35, wherein said control sample is obtained from a healthy subject not suffering or diagnosed with heart failure.

37. (canceled)

38. (canceled)

39. The method of claim 35, wherein the panel comprises hsa-miR-204, hsa-miR-199b-5p, hsa-miR-125a-5p, hsa-miR-143, and/or hsa-miR-195, and the comparison is a decrease in the measured levels of the miRNAs relative to the levels of the miRNAs in the control sample or the pre-determined reference levels.

40. (canceled)

41. The method of claim 35, wherein the panel comprises hsa-miR-16, hsa-miR-223, hsa-miR-320, hsa-miR-150, hsa-miR-378, hsa-miR-92a, hsa-miR-423-5p, hsa-miR-133a, hsa-miR-22, hsa-miR-21, hsa-miR-210, hsa-miR-20b, hsa-miR-499, hsa-miR-378*, hsa-miR-106b, hsa-miR-155, and/or hsa-miR-93, and the comparison is an increase in the measured levels of the miRNAs relative to the levels of the miRNAs in the control sample or the pre-determined reference levels.

42. (canceled)

43. (canceled)

44. A kit comprising miRNA-specific primers for reverse transcribing or amplifying two or more miRNAs selected from Table 1 in a plasma or serum sample from a patient having symptoms of heart failure and instructions for measuring said two or more miRNAs for predicting or assessing the level of severity of heart failure or heart failure progression in the patient.

45. The kit of claim 44, wherein the kit comprises a miRNA-specific primers for two or more miRNAs selected from hsa-miR-204, hsa-miR-199b-5p, hsa-miR-125a-5p, hsa-miR-143, hsa-miR-195, hsa-miR-16, hsa-miR-223, hsa-miR-320, hsa-miR-150, hsa-miR-378, hsa-miR-92a, hsa-miR-423-5p, hsa-miR-133a, hsa-miR-22, hsa-miR-21, hsa-miR-210, hsa-miR-20b, hsa-miR-499, hsa-miR-378*, hsa-miR-106b, hsa-miR-155, and hsa-miR-93.

46. A method for treating a patient for heart failure, the patient having elevated or low levels of one or more miRNAs listed in Table 1, comprising administering a miR-208a inhibitor or miR-208b inhibitor to the patient.

47. The method of claim 46, wherein the patient has elevated levels of miR-223, miR-16, miR-93, miR-106b, and/or miR-423-5p.

48. The method of claim 46, wherein the levels of miR-223, miR-16, miR-93, miR-106b and/or miR-423-5p are determined prior to treatment for heart failure.

49. The method of claim 46, wherein the level of miR-19b is monitored at least once during the course of treatment.
